(19) United States
(12) Patent Application Publication
(10) Pub. No.: US 2024/0042188 A1
(43) Pub. Date: Feb. 8, 2024

(54) COUPLING DEVICE FOR MEDICAL TUBING

(71) Applicant: CAREFUSION 303, INC., San Diego, CA (US)

(72) Inventors: Jason Andrew WINE, Brea, CA (US); Raymond P. FEITH, Chino Hills, CA (US); George MANSOUR, Diamond Bar, CA (US)

(21) Appl. No.: 18/341,060

(22) Filed: Jun. 26, 2023

Related U.S. Application Data
(60) Provisional application No. 63/395,277, filed on Aug. 4, 2022.

(51) Int. Cl. A61M 39/12; A61M 39/20

(52) U.S. Cl. CPC A61M 39/12; A61M 39/20

(57) ABSTRACT

A coupling device that is removably couplable with a mating connector is disclosed where the coupling device can include a housing and another structure that is movable relative to the housing such as an inner body or a cuff, and in a first configuration, the coupling device resists separation between the coupling device and a mating connector, in a second configuration, the resistance to separation between the mating connector and the coupling device is reduced relative to the first configuration such that the coupling device can permit separation between the coupling device and the mating connector when any of the inner body or the cuff is moved, biased, and/or flexed, and where the coupling device can be re-coupled with a mating connector after separation between the coupling device and the mating connector.

### CROSS REFERENCE TO RELATED APPLICATION

This application claims the benefit of priority under 35 U.S.C. § 119 to U.S. Provisional Patent Application Ser. No. 63/395,277, entitled “Coupling Device for Medical Tubing,” filed on Aug. 4, 2022, the entire contents of which is incorporated herein by reference.

### BACKGROUND

The present disclosure relates generally to medical fluid connectors and, more particularly, to coupling devices for connecting and disconnecting medical tubing.

Medical connections are widely used in fluid delivery systems such as those used in connection with intravenous (IV) fluid lines, blood access, hemodialysis, peritoneal dialysis, enteral feeding, drug vial access, and other procedures.

In some instances, the medical connection can become dislodged or disconnected in an unintended manner. For example, medical tubing of an IV set that is coupled to a catheter can become dislodged when an unintended or unexpected forces is exerted upon the catheter, which may exceed the design limitations of the catheter securement method. An unintended or unexpected force can be applied to the tubing and/or catheter when the patient moves or rolls over within a bed, or when the tubing or another portion of an intravenous set become caught on a portion of the bed, such as the railing, or when a patient is panicking, disoriented, or fidgeting to such an extent that the medical tubing is unintentionally or intentionally pulled away from the patient or away from the medical equipment coupled to the tubing.

### SUMMARY

In accordance with at least some embodiments disclosed herein is the realization that unintended dislodgement or disconnection of a medical connection, such as a medical fluid line, can result in injury to a patient or a caregiver, such as by depriving the patient of a medicament, increasing the potential for infection to the patient, and exposing the caregiver to harmful medicaments.

The present disclosure provides a coupling device for medical tubing, the coupling device comprising a housing having an inner surface forming a cavity, a first opening into the cavity, and a second opening into the cavity; and an inner body having a fluid passage, extending through the inner body, and a coupling sleeve, wherein the inner body is positioned within the cavity of the housing with the coupling sleeve extending toward the first opening of the housing such that the housing and the inner body are movable relative to each other; wherein, in a first position of the inner body, biasing of at least a portion of the coupling sleeve in a direction that is radially outward is resisted by engagement of the coupling sleeve with the housing; and wherein, in a second position of the inner body, the coupling sleeve is moved through the first opening in a direction that is away from the second opening of the housing such that the at least a portion of the coupling sleeve can be biased radially outward.

In some aspects, the present disclosure provides, a coupling device for medical tubing, the coupling device comprising a housing having a first end, a second end, and an inner surface forming a cavity, wherein the first end comprises a first opening into the cavity, and the second end comprises a second opening into the cavity; an inner body having a first end portion, a second end portion, a fluid passage extending through the first and second end portions of the inner body, the first end portion comprising a coupling sleeve formed by a wall having an inner surface that extends around a longitudinal axis defined by the fluid passage to form a recess extending from a first end of the inner body toward the second end portion, wherein the inner body is positioned within the cavity of the housing such that the housing and the inner body are movable relative to each other; and a biasing element positioned between the first end of the housing and the inner body; wherein, in a first position of the inner body, the first end of the inner body is longitudinally aligned with the first end of the housing such that biasing of at least a portion of the wall away from the longitudinal axis is resisted by the housing, and in a second position of the inner body, the second end of the housing is moved away from the second end portion of the inner body such that the first end of the inner body is longitudinally spaced apart from the first end of the housing and the at least a portion of the wall can be biased away from the longitudinal axis.

The present disclosure also provides a coupling device assembly comprising a housing having a first end, a second end, and an inner surface forming a cavity, wherein the first end comprises a first opening into the cavity, and the second end comprises a second opening into the cavity; an inner body having a first end portion, a second end portion, a fluid passage extending through the first and second end portions of the inner body, the first end portion comprising a coupling sleeve formed by a wall having an inner surface that extends around a longitudinal axis defined by the fluid passage to form a recess extending from a first end of the inner body toward the second end portion, wherein the inner body is positioned within the cavity of the housing such that the housing and the inner body are movable relative to each other; a biasing element positioned between the first end of the housing and the inner body; and a tubing extending through the second opening of the housing and into the cavity, wherein an end portion of the tubing is coupled to the fluid passage of the inner body, wherein, in a first position of the inner body, the first end of the inner body is longitudinally aligned with the first end of the housing such that biasing of at least a portion of the wall away from the longitudinal axis is resisted by the housing, and in a second position of the inner body, the second end of the housing is moved away from the second end portion of the inner body such that the first end of the inner body is longitudinally spaced apart from the first end of the housing and the at least a portion of the wall can be biased away from the longitudinal axis.

Methods of providing a coupling device for medical tubing are disclosed herein, including a method comprising inserting an inner body into a cavity of a housing such that the housing and the inner body are movable relative to each other, and a coupling sleeve defined by a proximal portion of the inner body extends through a first opening at a first end of the housing, inserting a biasing element into the housing such that the biasing element is positioned between a first end of the housing and the inner body, inserting a tubing through a second opening at a second end of the housing, and coupling the tubing to a fluid passage extending through the inner body, wherein, in a first position of the inner body, a first end of the inner body is longitudinally aligned with the first end of the housing such that biasing of at least a portion of coupling sleeve away from a longitudinal axis of the fluid passage of the inner body is resisted by the housing, and in a second position of the inner body, the second end of the housing is moved away from the second end of the inner body such that the first end of the inner body is longitudinally spaced apart from the first end of the housing and the at least a portion of the coupling sleeve can be biased away from the longitudinal axis.

Accordingly, the present application addresses several operational challenges encountered in prior fluid fitting connections and provides numerous improvements that enable the user to more easily and precisely connect and disconnect fluid fittings.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates a coupling device for medical tubing in use with an IV set coupled to a patient, in accordance with aspects of the present disclosure.

FIG. 2 illustrates a perspective view of an embodiment of a coupling device for medical tubing, in accordance with aspects of the present disclosure.

FIG. 3 illustrates a perspective view of the coupling device for medical tubing of FIG. 2, in accordance with aspects of the present disclosure.

FIG. 4 illustrates a cross-sectional view of the coupling device for medical tubing of FIG. 2, in accordance with aspects of the present disclosure.

FIG. 5 illustrates a cross-sectional view of the coupling device for medical tubing of FIG. 2 and a mating connector, in accordance with aspects of the present disclosure.

FIG. 6 illustrates a cross-sectional detail view of the coupling device for medical tubing of FIG. 4, in accordance with aspects of the present disclosure.

FIG. 7 illustrates a cross-sectional detail view of an embodiment of a coupling device for medical tubing, in accordance with aspects of the present disclosure.

FIGS. 8-10 illustrate cross-sectional views of the coupling device for medical tubing of FIG. 2 and a mating connector, in accordance with aspects of the present disclosure.

FIG. 11 illustrates a perspective view of an embodiment of a coupling device for medical tubing and a mating connector, in accordance with aspects of the present disclosure.

FIG. 12 illustrates a cross-sectional view of the coupling device for medical tubing of FIG. 11, in accordance with aspects of the present disclosure.

FIG. 13 illustrates a cross-sectional view of the coupling device for medical tubing of FIG. 11 and the mating connector, in accordance with aspects of the present disclosure.

FIGS. 14-16 illustrate perspective views of the coupling device for medical tubing of FIG. 11 and a mating connector, in accordance with aspects of the present disclosure.

FIGS. 17-19 illustrate cross-sectional views of the coupling device for medical tubing of FIG. 11 and a mating connector, in accordance with aspects of the present disclosure.

FIG. 20 illustrates a perspective view of an embodiment of a coupling device for medical tubing, in accordance with aspects of the present disclosure.

FIG. 21 illustrates a cross-sectional view of the coupling device for medical tubing of FIG. 20, in accordance with aspects of the present disclosure.

FIGS. 22-24 illustrate elevation views of the coupling device for medical tubing of FIG. 20 and a mating connector, in accordance with aspects of the present disclosure.

FIG. 25 illustrates a perspective view of an embodiment of a coupling device for medical tubing, in accordance with aspects of the present disclosure.

FIG. 26 illustrates a cross-sectional view of the coupling device for medical tubing of FIG. 25, in accordance with aspects of the present disclosure.

FIGS. 27 and 28 illustrate cross-sectional views of the coupling device for medical tubing of FIG. 25 and a mating connector, in accordance with aspects of the present disclosure.

FIGS. 29 and 30 illustrate cross-sectional detail views of an embodiment of a coupling device for medical tubing, in accordance with aspects of the present disclosure.

FIGS. 31-33 illustrate perspective views of the coupling device for medical tubing of FIG. 25 and a mating connector, in accordance with aspects of the present disclosure.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

In accordance with some embodiments, the present application discloses various features and advantages of a coupling device for medical tubing. The coupling device for medical tubing can provide for efficient and safe maintenance of fluid connections, such as the connections used for transferring medical fluids toward or away from a patient. The coupling device can maintain the fluid connection of a medical tubing by resisting disconnection when a pulling or tension force is applied to the coupling device, such as when a patient moves or when the medical tubing is pulled away from the patient. The coupling device also prevents injury to a patient or a caregiver by permitting disconnection of the fluid connection when a pulling or tension force exceeds a threshold. The coupling device also provides for efficient and safe reconnection of the fluid line by permitting the coupling device to be reconnected to the fluid transfer device, such as a catheter or needleless connector, thereby reestablishing the fluid pathway without requiring replacement of the coupling device. The coupling device can also include features to permit safe and efficient sterilization and cleaning for portions of the device before connection and reconnection therebetween.

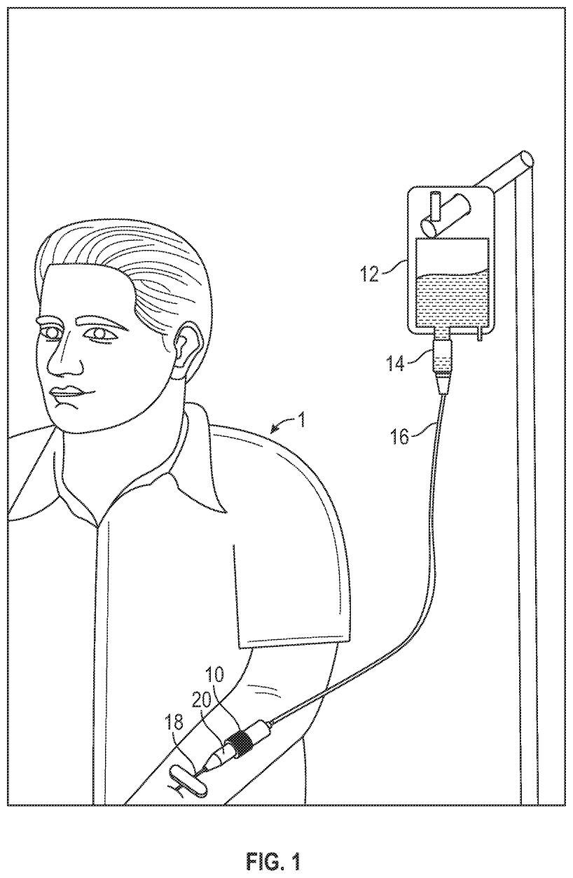

Referring now to the figures, FIG. 1 illustrates an embodiment of a coupling device for medical tubing in use in accordance with aspects of the present disclosure. The coupling device 10 is coupled with tubing of an IV set, which is being used to direct a fluid to a patient 1. The IV set can include a medicament bag 12, a drip chamber 14, tubing 16, and an IV catheter 18.

The coupling device 10 fluidly connects the tubing 16 to the IV catheter 18. Although the coupling device 10 is illustrated being coupled along a fluid pathway of an IV set, between a medicament bag 12 and a patient 1, it should be understood that the coupling device 10 can be connected within other fluid pathways, such as between a patient and an IV pump or between a patient and a dialysis machine. The coupling device 10 can also be connected along another portion of a fluid pathway. For example, the coupling device 10 can be connected along a proximal portion of the fluid pathway, such as being connected between the tubing 16 and the medicament bag 12 or other fluid therapy device.

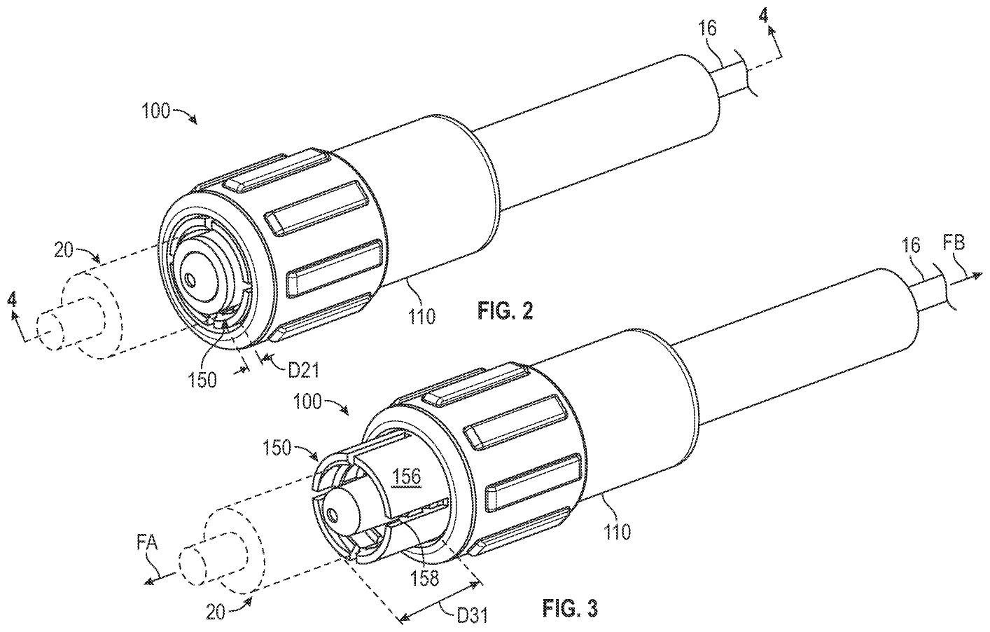

The coupling device 10 is configured to have a first configuration and a second configuration. In some embodiments of the present disclosure, the coupling device 10 can also have a third configuration. In the first configuration, the coupling device 10 can be coupled with a mating connector 20 to establish a fluid pathway between a tubing and the mating connector. The first configuration of an embodiment of a coupling device 100 is shown, for example, in FIG. 2. For clarity and brevity, the mating connector 20, is illustrated in a general manner with broken lines.

The coupling device 100 includes a housing 110 and an inner body 150 positioned, at least partially, within an inner cavity of the housing 110. The inner body 150 includes a portion forming a coupling sleeve configured to couple with the mating connector 20. The coupling sleeve can include any structure configured to couple with a mating connector 20, such as a thread, a barb, or an interference fit between the inner body 150 and the mating connector 20.

When the coupling device 100 is in the first configuration, the inner body 150 is in a first position within the cavity of the housing 110, whereby the coupling sleeve of the inner body 150 resists separation or disconnection of the mating connector 20 from the coupling device 100.

In some embodiments of the present disclosure, when the inner body 150 is in the first position, an end of the inner body is approximately aligned or coplanar with an end of the housing. In some embodiments, a portion of the coupling sleeve of the inner body 150 or another portion of the inner body 150 may extend through the end of the housing by a first distance D21 when the inner body 150 is in the first position.

When the coupling device 100 is in the first configuration, with the inner body 150 in the first position, the coupling device 100 can resist unintended separation of the mating connector 20 from the coupling device 100 by resisting biasing or flexing of the coupling sleeve away from mating connector 20. In some embodiments of the present disclosure, biasing of the coupling sleeve radially outward is resisted by the housing 110, such that, when the mating connector 20 and the coupling device 100 are pulled away from each other, the sleeve will engage against a portion of the housing 110, thereby resisting movement of the mating connector 20 out of the sleeve.

The coupling device 100 can be moved from the first configuration to the second configuration, which is illustrated in FIG. 3, when a pulling or tension force exceeds a threshold of the coupling device 100. For example, when a patient moves in an unintended manner, or when the medical tubing is pulled away from the patient, a pulling or tension force FA, FB may be applied to the mating connector 20 or the tubing 16 in a direction away from the coupling device 100. In at least some instances of the present disclosure, a pulling or tension force FA is in a distal direction relative to the coupling device, and a pulling or tension force FB is in a proximal direction.

If the pulling or tension force FA, FB exceeds a first threshold of the coupling device 100, any of the housing 110 and the inner body 150 can move away from each other so that the inner body 150 moves from the first position to a second position. The movement of the housing 110 and the inner body 150, relative to each other, can be along a longitudinal axis of the inner body 150 that extends between the mating connector 20 and an end portion of the tubing 16 connected to the coupling device 100.

In the second position of the inner body 150, at least a portion of the sleeve extends out of the cavity and away from an end of the housing 110 by a second distance D31. The second distance is greater than the first distance between the distal end of the sleeve and the end of the housing when the inner body 150 is in the first position.

If a pulling or tension force FA, FB exceeds a second threshold of the coupling device 100, the coupling sleeve of the inner body 150 is configured such that the engagement of the mating connector 20 against the sleeve can cause at least a portion of the sleeve to bias or flex away from the mating connector 20. When the coupling sleeve flexes away from the mating connector 20, the resistance to separation of the coupling device 100 from the mating connector 20 is reduced, relative to when the coupling device 100 is in the first configuration, thereby permitting the coupling device 100 to separate from the mating connector 20. In some embodiments, engagement of a thread of the mating connector 20 against a thread of the inner body 150 when the force FA, FB exceeds the second threshold causes the portion of the sleeve to flex away from the mating connector 20. In some aspects of the present disclosure, a thread of the coupling device 100 can skip over or pass a thread of the mating connector 20 when the force FA, FB exceeds a second threshold of the coupling device 100.

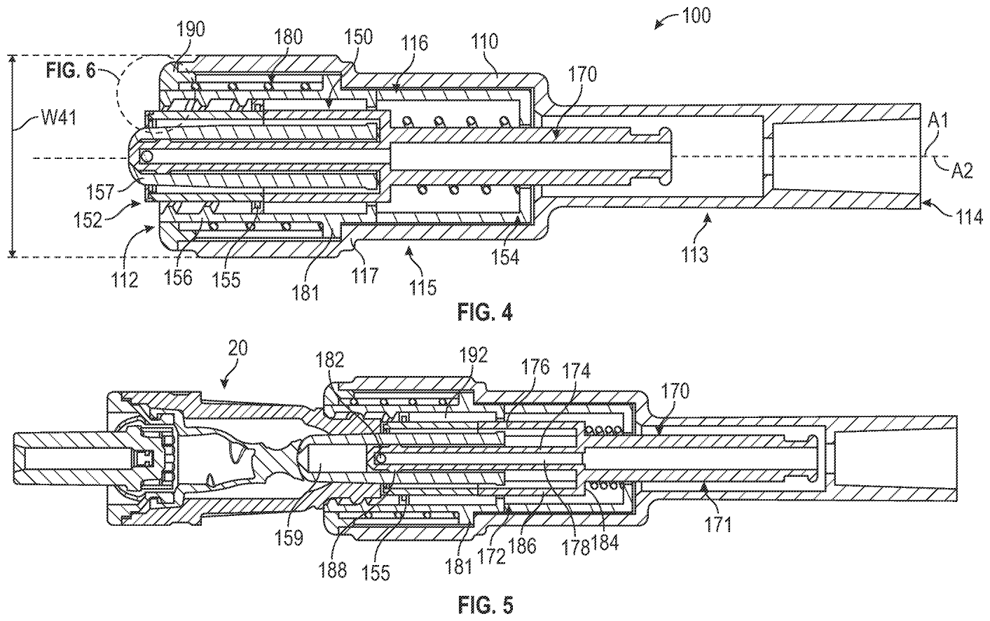

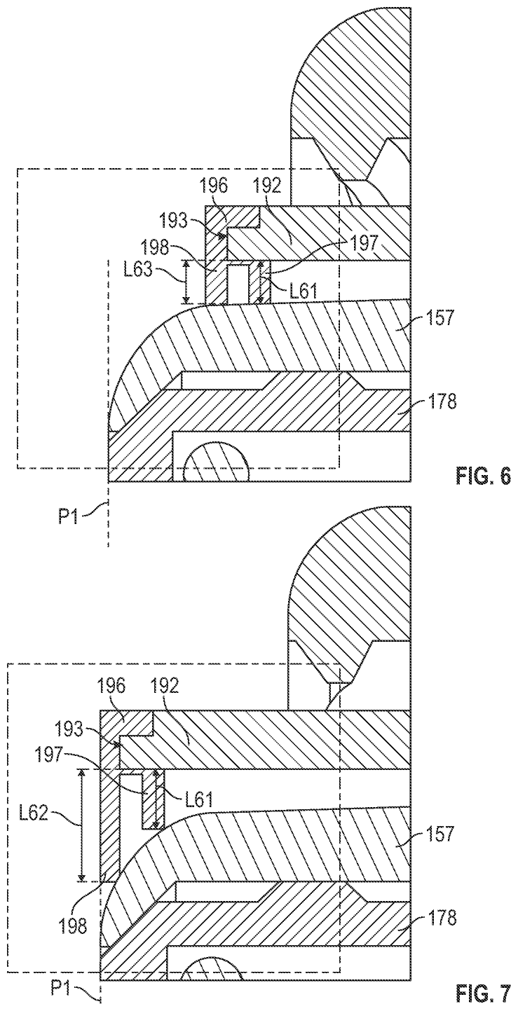

FIG. 4 illustrates a cross-sectional elevation view of the coupling device 100. The coupling device 100 includes a housing 110, an inner body 150, and an inner body biasing element 180. The inner body 150 is configured to resist and/or permit separation of a mating connector 20 from the coupling device. In some aspects of the present disclosure, separation of a mating connector 20 from the coupling device is resisted when the inner body is in a first position, and separation of a mating connector 20 from the coupling device is permitted when the inner body is in a second position. The first position of the inner body 150 is shown, for example, in FIGS. 4 and 5, and the second position of the inner body 150 is shown, for example, in FIGS. 6 and 7.

The inner body 150 is located within a cavity formed by the inner surface of the housing 110 and is movable between the first and second positions. The inner body 150 can be positioned within a location of the cavity along a distal end portion of the housing. An inner body biasing element 180 can also be positioned within the cavity to bias the inner body 150 in a proximal direction to resist separation of a mating connector 20 from the coupling device 100.

The housing 110 includes a proximal end portion 113 forming a first end 114, a distal end portion 115 forming a second end 112. The first end 114 is opposite to the second end 112, and a longitudinal axis A1 is defined between the first and second ends of the housing. An inner surface 116 of the housing forms a cavity 118 that extends between the first end 114 and the second end 112, and is configured to receive the inner body 150 therein.

The first end 114 of the housing, or a portion of the housing adjacent to the first end 114, forms a first opening of the housing which is configured permit tubing to be positioned through the first opening into the cavity. The second end 112 of the housing, or a portion of the housing adjacent to the second end 112, forms a second opening of the housing which is configured to permit the inner body 150 to be inserted through the second opening into the cavity.

In some embodiments of the present disclosure, the inner surface 116 of the housing forms a ridge 117 that extends between the first end 114 and the second end 112 of the housing. In some aspects of the present disclosure, a portion of the inner body 150 is positioned between the ridge 117 and the second end 112 of the housing, and the inner body 150 is longitudinally movable between the ridge 117 and the second end 112 of the housing

In embodiments of the present disclosure, the inner surface 116 of the housing forms any of a groove and/or ridge, and the inner body 150 forms the other of a groove and/or ridge, where the groove and/or ridge of the housing and inner body can extend in a longitudinal direction that is approximately parallel to a longitudinal axis A1 of the housing.

The groove and/or ridge of the housing 110 is configured to mate with or engage against the inner body 150 such that, when the housing 110 is rotated around the longitudinal axis A1, the portion of the housing 110 forming the groove and/or ridge can engage against the portion of the inner body 150, thereby rotating the inner body 150 with the housing 110. By rotationally coupling the housing 110 and the inner body 150, the housing 110 can be rotated to engage threads of the inner body 150 with complementary threads of a mating connector 20.

The inner body 150 has a first end portion 154, a second end portion 152, and a passage 159 extending through the first and second end portions of the inner body. The second end portion 152 of the inner body forms a coupling sleeve 156. The coupling sleeve 156 has an inner surface that extends around a longitudinal axis A2 defined between the first and second end portions of the inner body, where the inner surface forms a recess extending through the second end portion 152 of the inner body toward the first end portion 154 thereof.

The coupling sleeve 156 can have a discontinuous circumference configured to permit the coupling sleeve to flex or bias toward or away from the longitudinal axis A2. The discontinuous surface can be formed by a channel 158 that extends through the coupling sleeve 156 in a direction from a second end of the inner body toward the first end portion 154 of the inner body.

In some embodiments of the present disclosure, two or more channels 158 extend through the coupling sleeve 156 of the inner body to form a plurality of flexible fingers, wherein each finger is defined between adjacent channels 158. As such, the inner body 150 can include a first flexible finger and a second flexible finger. In some aspects of the present disclosure, the inner body 150 includes four flexible fingers, which are separated from each other by three channels through the coupling sleeve 156.

The second end portion 152 of the inner body, along the one or more channel 158, is more flexible, relative to another portion of the inner body that is spaced apart from the second end portion 152. In some embodiments, the second end portion 152 of the inner body is more flexible than the first end portion 154 of the inner body.

The second end portion 152 of the inner body also forms a luer protrusion 157 configured to engage against a mating connector 20 and form a portion of a fluid passageway through the coupling device 100.

The luer protrusion 157 is formed by the second end portion 152 of the inner body and can have an outer surface forming a male luer shape. The structure and orientation of the luer protrusion 157 is such that the luer protrusion extends through the recess defined by the coupling sleeve 156 which extends around a perimeter or circumference of the luer protrusion 157.

In some embodiments of the present disclosure, an inner surface of the coupling sleeve 156 is spaced apart from the outer surface of the luer protrusion 157. The space between the coupling sleeve 156 and the luer protrusion 157 can permit a mating connector 20 to be affixed to the coupling device 100 with a portion of the mating connector 20 positioned between the coupling sleeve 156 and the luer protrusion 157. In some aspects of the present disclosure, the luer protrusion 157 is configured to displace a valve of the mating connector 20.

The inner surface of the luer protrusion forms a portion of the valve passage 159 through the inner body 150 and along the second end portion 152 thereof. The valve passage 159 can permit a fluid to through the coupling device 100 when coupled with a mating connector 20 in any of the first and second positions of the inner body 150.

An inner body biasing element 180 is positioned in the cavity of the housing 110 to direct or bias the inner body 150 toward the first position. To bias the inner body 150 toward the first position, or in a direction toward the first end 114 of the housing, the biasing element 180 can be positioned between the inner body 150 and the second end 112 of the housing.

The inner body biasing element 180 can be any of a spring, a resilient bellows, or any similar structure configured to move the inner body 150. A first end of the inner body biasing element 180 can engage against the inner body 150, and a second end of the inner body biasing element 180 can engage against a portion of the housing 110 at the second end 112 thereof.

In some embodiments of the present disclosure, a portion of the inner body 150 forms a flange 181 configured to engage against the inner body biasing element 180. The flange 181 is positioned along the second end portion 152 of the inner body and extends radially outward in a direction away from the longitudinal axis A2 of the inner body. The flange 181 can extend around the perimeter of the inner body 150 or can be formed by a series of discontinuous flanges 181 that are separated from each other around the perimeter of the inner body 150.

When the inner body 150 is positioned within the cavity of the housing 110, a first end of the inner body biasing element 180 can engage against the flange 181, and a second end of the inner body biasing element 180 can engage against the housing 110. The biasing element 180 exerts a force on the flange 181 of the inner body, thereby biasing the inner body 150 in the proximal direction toward the first end 114 of the housing.

In some embodiments of the present disclosure, the ridge 117 of the housing is configured to resist movement of the inner body 150 toward the first end 114 of the housing. When the inner body 150 is in the first position, the flange 181 of the inner body can engage against the ridge 117 of the housing flange 120, and when the inner body 150 is in the second position, the flange 181 of the inner body is spaced apart from the ridge 117 of the housing.

In some embodiments of the present disclosure, a cap 190 is coupled to the second end 112 of the housing to retain the inner body biasing element 180 and the inner body 150 within the cavity of the housing. In such embodiments, the second end of the inner body biasing element 180 can engage against the cap 190.

The cap 190 includes a passage defining at least a portion of the second opening through the second end 112 of the housing. The passage through the cap 190 can have a cross-sectional width that is less than a cross-sectional width formed by the cavity of the housing such that the cap 190 can resist movement of the inner body biasing element 180 out of the cavity and permit the inner body to move out of the cavity through the passage of the cap 190.

In some aspects of the present disclosure the cap 190 can be unitarily formed with the housing 110 to form a ridge or flange of the housing that extends radially inward toward the longitudinal axis A1 of the housing.

To permit or resist a fluid flow through the luer protrusion, the coupling device 100 can include a valve 170. The valve 170 is positioned in the cavity of the housing 110, and is movable between the first end 114 and the second end 112 of the housing. The valve 170 can also be configured to move relative to the housing 110 and to the inner body 150, such that the valve 170 is movable when the inner body 150 is in any of the first and second positions.

The valve 170 is movable between a closed position and an open position. In the closed position, the valve 170 can resist movement of a fluid through the coupling device 100. In some embodiments of the present disclosure, in the closed position, the valve 170 obstructs the valve passage 159 to resist movement of a fluid therethrough. In the open position, the valve 170 is moved, relative to the inner body 150, to permit a fluid to flow through the inner body 150 and the valve 170. The closed and open positions of the valve 170 are shown, for example, in FIGS. 4 and 5, respectively.

The valve 170 includes a proximal end portion 171 and a distal end portion 172, where the distal end portion forms a post 174 and an arm 176. An inner surface of the valve 170 defines a fluid channel 178 extending through the proximal and distal end portions of the valve 170. In some aspects of the present disclosure, a portion of the fluid channel is formed by an opening 182 that extends radially between the inner and outer surfaces of the post 174.

The arm 176 of the valve is configured to engage against a mating connector 20 such that the valve 170 moves in a direction from the closed position toward the open position when the mating connector 20 is coupled to the coupling device 100.

The arm 176 extends along the post 174 and in a direction away from the proximal end portion 171 of the valve. The arm 176 can include a first segment 184 that extends radially outward in a direction away from the outer surface of the post, and a second segment 186 that extends from the first segment in a direction along the post and in direction away from the proximal end portion 171 of the valve.

In some embodiments of the present disclosure, the first segment 184 extends radially outward along a portion of the valve 170 between the proximal and distal end portions 171, 172. The first segment 184 comprises a length that is greater than a thickness of the arm 176 such that the inner surface of the arm 176 is spaced apart from the outer surface of the post 174.

The valve 170 is positioned in the cavity of the housing 110 with the post 174 extending into the valve passage 159 of the luer protrusion and the arm positioned extends between the outer surface of the luer protrusion and the inner surface of the coupling sleeve. The proximal end portion 171 of the valve extends through the passage 159 formed through the first end portion 154 of the inner body.

The outer surface of the post 174 defines a cross-sectional width that is less than a cross-sectional width defined by the inner surface of the luer protrusion 157 such that a gap is formed therebetween. The gap can permit a fluid between the outer surface of the post 174 and the inner surface of the luer protrusion 157, and through the opening 182.

In some embodiments, the post 174 comprises a protrusion 188 that extends radially outward to engage against the inner surface of the luer protrusion 157. The protrusion 188 can be configured to engage against the luer protrusion to resist movement of the post within the valve passage 159 in a direction that is non-linear or transverse relative to the longitudinal axis A2. In some aspects, the protrusion 188 can resist non-linear or transverse movement of the post 174 when the valve moved between the open and closed positions.

In the closed position of the valve 170, the post 174 obstructs or intersects an opening through the luer protrusion 157 to resist a fluid flow therethrough. When the valve 170 is moved to the open position, the post 174 is spaced apart from the opening of the luer protrusion 157, relative to when the valve 170 is in the closed position, to permit a fluid to move through the valve passage 159 of the luer protrusion and through the valve 170.

The arm 176, in some embodiments of the present disclosure, includes a cylindrical extension 192 that extends from the arm 176 in a direction away from the first end 114 of the housing. The cylindrical extension 192 can be configured to direct a force to arm 176 such that the valve 170 moves relative to the inner body 150 when the coupling device 100 is coupled to a mating connector. The cylindrical extension 192 can also be configured to engage against the luer protrusion of the inner body to form a seal between the cylindrical extension 192 and the luer protrusion 157. The seal formed between the cylindrical extension 192 and the luer protrusion 157 can resist unintended materials, such as fluids and/or solids, from moving into or out of the cavity of the housing 110.

The cylindrical extension 192 can extend from the second segment 186 of the arm to a terminal end 193 positioned proximal to the distal end of the luer protrusion 157. The cylindrical extension 192 and arm 176 can define a length between the first segment 184 and the terminal end 193 of the cylindrical extension.

In some embodiments of the present disclosure, the length of the cylindrical extension 192 and arm 176 are configured such that the terminal end 193 of the cylindrical extension is proximal to the distal end of the luer protrusion 157, and the distal most portion of the luer protrusion 157 extends beyond the terminal end 193 of the cylindrical extension, as shown in the detail view of FIG. 6. In such an embodiment, the terminal end 193 of the cylindrical extension is spaced apart from a plane P1 defined by the distal end of the luer protrusion 157.

In some embodiments of the present disclosure, the length of the cylindrical extension 192 and arm 176 are configured such that the terminal end 193 of the cylindrical extension is coplanar with or aligned with the plane P1 defined by the distal end of the luer protrusion 157, as shown in the detail view of FIG. 7.

In some embodiments of the present disclosure, the seal between the arm 176 and the luer protrusion 157 is formed by a valve seal 196. The valve seal 196 is coupled to any of the arm 176 and the cylindrical extension 192, and is configured to engage against the outer surface of the luer protrusion 157. The valve seal 196 is engaged against the outer surface of the luer protrusion 157 to maintain a seal as the valve 170 is moved between the closed and open positions.

The valve seal 196 is coupled to the terminal end 193 of the cylindrical extension and extends radially inward in a direction toward the outer surface of the luer protrusion 157. The present disclosure contemplates that the valve seal 196 can be coupled to a portion of the arm 176 and/or the cylindrical extension 192 that is proximal to or spaced apart from the terminal end 193 of the cylindrical extension.

In some embodiments of the present disclosure, the valve seal 196 can include a proximal layer 197 and a distal layer 198, where each of the proximal and distal layers 197, 198 extend in a direction away from the cylindrical extension 192. Each of the proximal and distal layers 197, 198 comprise a length extending in a direction from the cylindrical extension 192 to an end of each respective layer. In some embodiments, such as is shown in FIG. 6, for example, the length L61 of the proximal layer 197 is approximately equal to the length L62 of the distal layer 198. In some embodiments, such as is shown in FIG. 7, for example, the length L61 of the proximal layer 197 is less that the length L63 of the distal layer 198.

In some embodiments of the present disclosure, an inner body seal 155 forms a seal between the inner body 150 and the valve 170 to resist unintended materials, such as fluids and/or solids, from moving into or out of the cavity of the housing 110.

To maintain a seal between the inner body 150 and the valve 170, the inner body seal 155 is configured to move relative to any of the inner body 150 and valve 170. The inner body seal 155 is coupled to an inner surface of the coupling sleeve 156 and is configured to engage against any of the arm 176 and the cylindrical extension 192 to form the seal when valve 170 moves between the open and closed positions.

Although the inner body seal 155 can be coupled to the inner body 150, it should be understood that the present disclosure contemplates that the inner body seal 155 can be coupled to the valve 170 in some embodiments.

The proximal end portion 171 of the valve can be fluidly coupled to the tubing 16 to permit a fluid flow through the fluid channel 178 and tubing 16. The tubing 16 extend through the first end 114 of the housing and be coupled to the proximal end portion 171 of the valve. In some embodiments, another length of tubing or a bellows is positioned between the proximal end portion 171 of the valve and the first end 114 of the housing, and the tubing 16 can be coupled to the first end 114 of the housing.

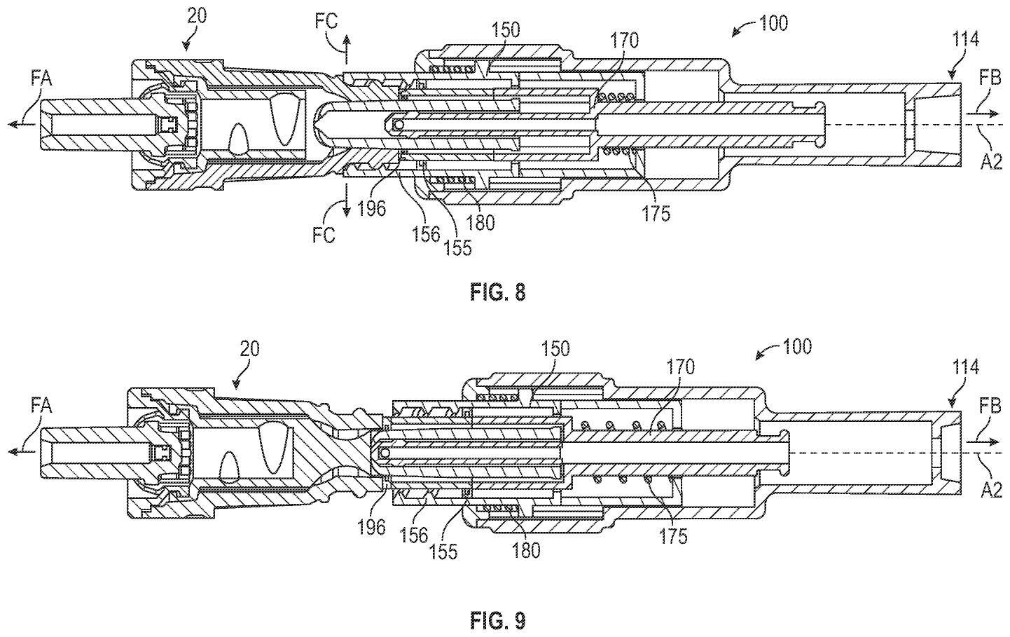

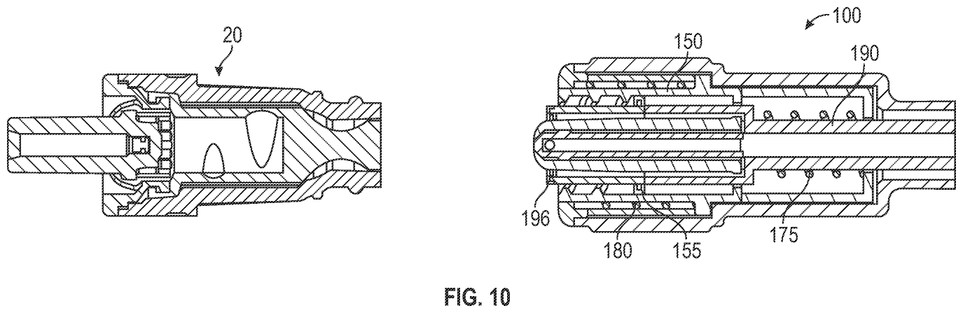

The coupling device 100 and a mating connector 20 are shown together in FIGS. 8-10, where the coupling device 100 is in the second configuration in FIGS. 8 and 9, and in the first configuration in FIG. 10.

The coupling device 100 is moved to the second configuration after a pulling or tension force FA, FB exceeds a first threshold of the coupling device 100. When the mating connector 20 is coupled with the coupling device 100, and a pulling or tension force exceeds a threshold of the coupling device 100, the mating connector 20 moves with the inner body 150 relative to the housing 110. As the coupling device 100 is moved to the second configuration, the inner body 150 is moved in a direction away from the first end 114 of the housing, and the inner body biasing element 180 is compressed between the inner body 150 and the housing 110, as shown in FIG. 8.

In the second configuration, the mating connector 20 can remain coupled to the inner body 150 such that the valve 170 remains in the open position and a fluid can flow between the mating connector 20 and the coupling device 100. In some embodiments of the present disclosure, a valve biasing element 175 is compressed between the valve 170 and a portion of the inner body 150 when the valve is in the open position.

If the pulling or tension force FA, FB exceeds a second threshold of the coupling device 100, the engagement of the mating connector 20 against the coupling sleeve 156 of the inner body causes at least a portion of the coupling sleeve 156 to bias or flex in a direction FC away from the longitudinal axis A2. In some embodiments of the present disclosure, when the second threshold is exceeded, a thread of the mating connector 20 can skip over or pass a thread of the coupling device 100, thereby reducing the resistance to separation of the coupling device 100 from the mating connector 20.

When the mating connector 20 separates from the coupling device 100, the valve 170 can move from the open position (FIG. 8) to the closed position such that the valve passage 159 is obstructed and movement of a fluid therethrough is resisted, as shown in FIG. 9. In some embodiments, the valve biasing element 175 expands to exert a force on the valve 170 to move the valve from the open position toward the closed position.

After the pulling or tension force FA, FB is reduced to less than the first threshold, the inner body 150 can move from the second position to the first position so that the coupling device 100 is in the first configuration as shown in FIG. 10. The inner body 150 can move to the first position so that the coupling device 100 is in the first configuration when the pulling the tension force FA, FB is reduced or when the mating connector 20 is separated from the coupling device 100 such that the pulling the tension force FA, FB is no longer present.

The inner body biasing element 180 expands to exert a force on the inner body 150 such that the inner body moves from the second position to the first position. The inner body biasing element 180 is positioned between the second end 112 of the housing and the flange 181 of the inner body 150, and extends along the second end portion 152 of the inner body. However, in some embodiments of the present disclosure, the inner body biasing element 180 can positioned along another portion the inner body 150.

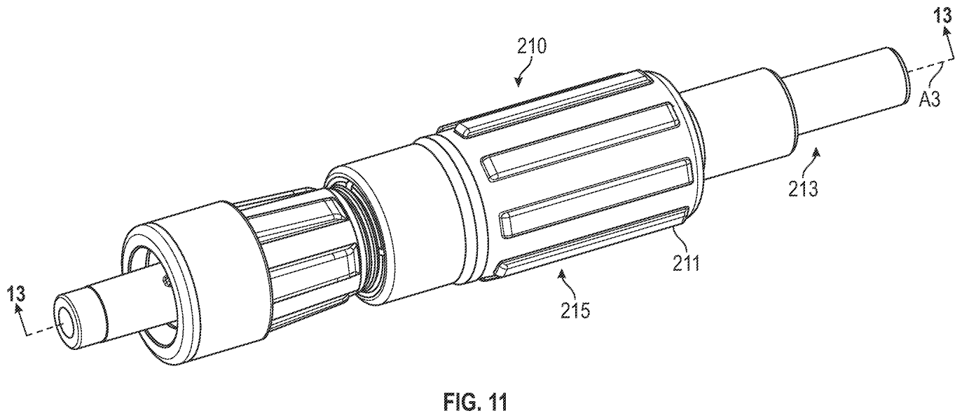

In some embodiments of the present disclosure, the inner body biasing element 180 is positioned along the first end portion 154 of the inner body. An example of the inner body biasing element 180 positioned along the first end portion 154 of the inner body is show in the coupling device 200 of FIGS. 11-19. The coupling device 200 can include features that are the same or similar to the coupling device 100, therefore, for the sake of clarity and brevity, detailed description for same or similar features are not repeated.

Referring to FIGS. 11-13, the coupling device 200 includes a housing 210 having an outer surface 211 defining a cross-sectional width W121 of the housing 210, where the cross-sectional width W121 of the housing 210 is less than a cross-sectional width W41 of the housing 110 of the coupling device 100.

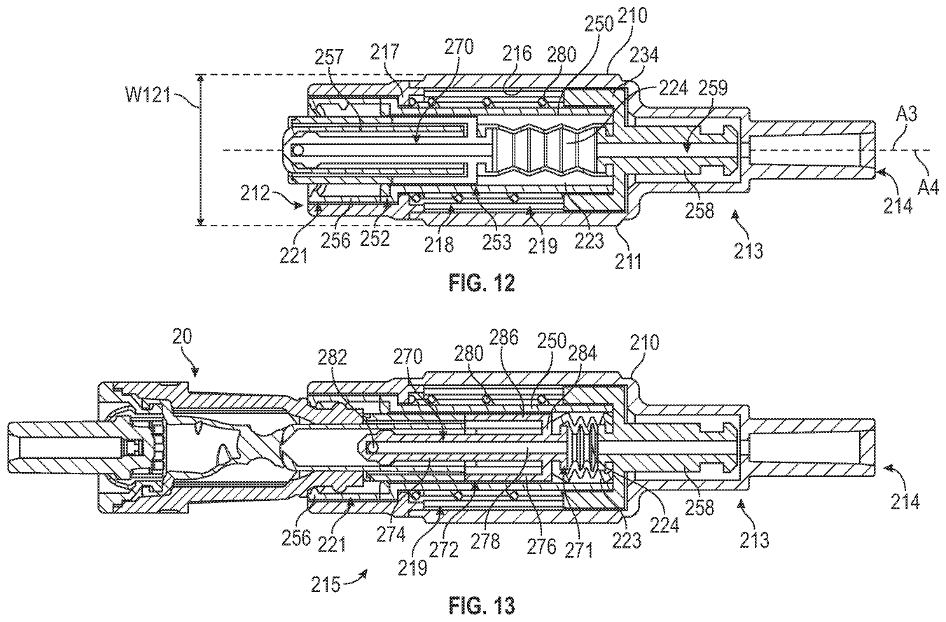

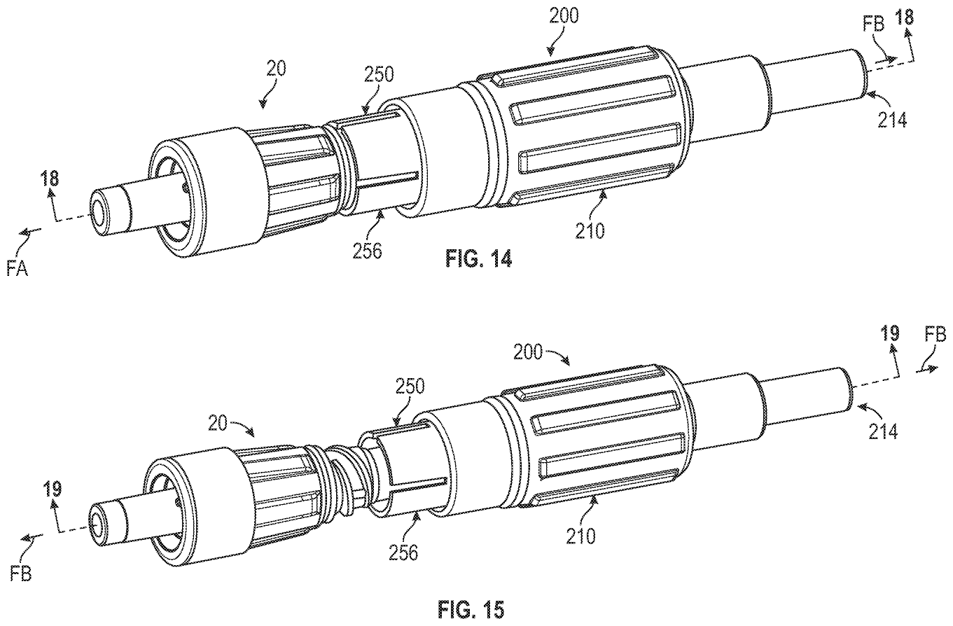

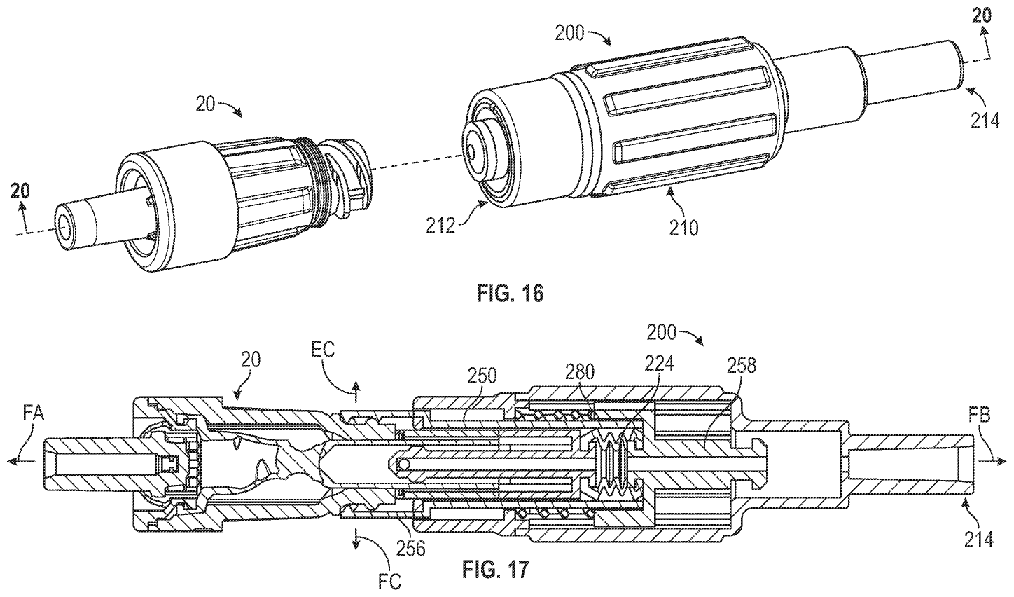

FIG. 12 illustrates a cross-sectional elevation view of the coupling device 200. The coupling device 200 includes a housing 210, an inner body 250, and an inner body biasing element 280. The inner body 250 is configured to resist and/or permit separation of a mating connector 20 from the coupling device. In some aspects of the present disclosure, separation of a mating connector 20 from the coupling device is resisted when the inner body is in a first position, and separation of a mating connector 20 from the coupling device is permitted when the inner body is in a second position. The first position of the inner body 150 is shown, for example, in FIGS. 13 and 16, and the second position of the inner body 150 is shown, for example, in FIGS. 14 and 17.

The inner body 250 is movable between the first and second positions by positioning the inner body 250 within a cavity formed by the inner surface of the housing 210. The inner body 250 can be positioned within the cavity along a distal end portion of the housing. The inner body biasing element 280 is positioned within the cavity to bias the inner body 250 in a proximal direction to resist separation of a mating connector 20 from the coupling device 200.

The housing 210 has a proximal end portion 213 forming a first end 214, a distal end portion 215 forming a second end 212. The first end 214 is opposite to the second end 212, and a longitudinal axis A3 is defined between the first and second ends. An inner surface 216 of the housing forms a cavity 218 that extends between the first end 214 and the second end 212 of the housing and is configured to receive the inner body 250 therein.

The first end 214 of the housing, or a portion of the housing adjacent to the first end 214, forms a first opening of the housing which is configured permit tubing to be positioned through the first opening into the cavity. The second end 212 of the housing, or a portion of the housing adjacent to the second end 212, forms a second opening of the housing which is configured to permit the inner body 250 to be inserted through the second opening into the cavity.

The outer surface 211 of the housing defines the cross-sectional width W121 of the housing, where the cross-sectional width W121 does not increase along the distal end portion 215.

The inner surface 216 of the housing forms a ridge 217 that extends between the first end 214 and the second end 212 of the housing. The ridge 217 is configured with a proximal portion at the inner surface of the housing and a distal end that extends away from the inner surface 216 of the housing, such that the ridge 217 forms a cantilever structure extending into the cavity 218. The cantilever-shaped ridge 217 comprises a first end surface that faces toward the first end 214 of the housing, and a second end surface that faces toward the second end 212 of the housing.

The cantilever-shaped ridge 217 also defines a proximal portion 219 of the cavity 218 and a distal portion 221 of the cavity 218, where a cross-sectional width of the proximal and distal portions of the cavity 218 are greater than a cross-sectional width of the cavity along the ridge 217.

In some aspects of the present disclosure, a portion of the inner body 250 is positioned between the ridge 217 and the second end 212 of the housing, and the inner body 250 is longitudinally movable between the ridge 217 and the second end 212 of the housing.

The inner body 250 includes features corresponding to the coupling sleeve 156 of the coupling device 100, except where disclosed herein.

The second end portion 252 of the inner body forms a luer protrusion 257, and the first end portion 254 forms boss 258. A passage 259 of the inner body extends through the luer protrusion 257 and the boss 258, such that a fluid can flow through the inner body. Between the luer protrusion 257 and the boss 258, an inner surface of the inner body 250 forms a cavity 223 such that the boss 258 is spaced apart from the luer protrusion 257.

Between the second end portion 252 and the first end portion 254, the outer surface of the inner body 150 can have middle portion 253 with a cross-sectional width that is less than the cross-sectional width along the first and second end portions 252, 254. The middle portion 253 permits the inner body 150 to be positioned within the cavity of the housing with the ridge 217 positioned along the middle portion 253 such that the inner body is movable relative to the housing 210.

The inner body 250 is movable between a first position in which the ridge 217 is proximal to the second end portion 252, and a second position in which the inner body 250 is moved in a direction away from the first end 214 of the housing and the ridge 217 is proximal to the first end portion 254.

The inner body biasing element 280 is positioned within the cavity 218 of the housing and extends along the middle portion 253 of the inner body. The biasing element 280 is configured to direct or bias the inner body 150 in a direction toward the first position.

To bias the inner body 250 toward the first position, or in a direction toward the first end 114 of the housing, the biasing element 280 is positioned between the distal end portion 215 of the housing and the first end portion 254 of the inner body.

The inner body biasing element 280 can be any of a spring, a resilient bellows, or any similar structure configured to direct the inner body 250. A first end of the inner body biasing element 280 can engage against the first end portion 254 of the inner body, and a second end of the inner body biasing element 280 can engage against the ridge 217.

In some embodiments of the present disclosure, the ridge 217 of the housing is configured to resist movement of the inner body 250 toward the first end 214 of the housing. When the inner body 250 is in the first position, the second end portion 252 of the inner body can engage against the ridge 217 of the housing, and when the inner body 250 is in the second position, the second end portion 252 of the inner body is spaced apart from the ridge 217 of the housing.

To permit or resist a fluid flow through the luer protrusion, the coupling device 200 includes a valve 270. The valve 270 includes features corresponding to the valve 170 of the coupling device 100, excepts where disclosed herein.

The valve 270 is positioned in the cavity of the housing 210, and is movable between the first end 214 and the second end 212 of the housing. The valve 270 can also be configured to move relative to the housing 210 and to the inner body 250, such that the valve 270 is movable when the inner body 250 is in any of the first and second positions.

The valve 270 is movable between a closed position and an open position. In the closed position, the valve 270 can resist movement of a fluid through the coupling device 200. In some embodiments of the present disclosure, in the closed position, the valve 270 obstructs the passage 259 along the luer protrusion to resist movement of a fluid therethrough. In the open position, the valve 270 is moved, relative to the inner body 250, to permit a fluid to flow through the inner body 250 and the valve 270. The closed and open positions of the valve 270 are shown, for example, in FIGS. 12 and 13, respectively.

The valve 270 includes a proximal end portion 271 and a distal end portion 272, where the distal end portion forms a post 274 and an arm 276. An inner surface of the valve 270 defines a fluid channel 278 extending through the proximal and distal end portions of the valve 270. In some aspects of the present disclosure, a portion of the fluid channel is formed by an opening 282 that extends radially between the inner and outer surfaces of the post 274.

The arm 276 of the valve extends along the post 274, and includes a first segment 284 that extends radially outward in a direction away from the outer surface of the post, and a second segment 286 that extends from the first segment in a direction along the post and in direction away from the proximal end portion 271 of the valve.

The valve 270 is positioned in the cavity of the housing 210 with the post 274 extending into the passage 259 of the luer protrusion and the arm positioned extends between the outer surface of the luer protrusion and the inner surface of the coupling sleeve. The proximal end portion 271 of the valve extends toward the first end portion 254 of the inner body and terminates in the cavity 223.

Although a fluid can flow through the passage 259 and the cavity 223 of the inner body and passage 259 during use of the coupling device 200, the cavity 223 is configured to receive a bellows 224 therein. The bellows 224 can have a first end opening coupled to the boss 258 and fluidly coupled with the passage 259 of the first end portion 254 of the inner body, and a second end opening coupled to the luer protrusion 257 and fluidly coupled with the passage 259 of the second end portion 252 of the inner body.

The bellows 224 can direct a fluid moving between the a fluid channel 278 of the valve and the passage 259 of the inner body when the valve 270 is in the closed and open positions. In some embodiments, the bellows 224 comprises a first length when the valve 270 is in the closed position, and a second length when the valve 270 is in the open position, wherein the second length is less than the first length. In some aspects of the present disclosure, the bellows is in an extended configuration when the valve 270 is in the closed position, and is compressed when the valve 270 is in the open position.

Although a bellows is disclosed by the present disclosure, it should be understood that other structures are contemplated, which can form a fluid pathway between the valve 270 and the passage 259 of the first end portion 254 of the inner body. For example, a flexible length of tubing can extend between the valve 270 and the boss 258, and the tubing can be configured such that a portion between the valve 270 and the boss 258 bends or deflects when the valve 270 is in the open position.

The coupling device 200 and a mating connector 20 are shown together in FIGS. 14-17, where the coupling device 200 is in the second configuration in FIGS. 14 and 15, and in the first configuration in FIG. 16. FIGS. 17-19 are cross-sectional views of the coupling device 200 and a mating connector 20 of FIGS. 14-17, respectively.

The coupling device 200 is moved to the second configuration after a pulling or tension force FA, FB exceeds a first threshold of the coupling device 200. When the mating connector 20 is coupled with the coupling device 200, and a pulling or tension force exceeds a threshold of the coupling device 200, the mating connector 20 moves with the inner body 250 relative to the housing 210. As the coupling device 200 is moved to the second configuration, the inner body 250 is moved in a direction away from the first end 214 of the housing, and the inner body biasing element 280 is compressed between the inner body 250 and the housing 210, as shown in FIGS. 14 and 17.

In the second configuration, the mating connector 20 can remain coupled to the inner body 250 such that the valve 270 remains in the open position and a fluid can flow between the mating connector 20 and the coupling device 200. In some embodiments of the present disclosure, the bellows 224 is compressed between the valve 270 and the boss 258 when the valve is in the open position.

If the pulling or tension force FA, FB exceeds a second threshold of the coupling device 200, the engagement of the mating connector 20 against the coupling sleeve 256 of the inner body causes at least a portion of the coupling sleeve 256 to bias or flex in a direction FC away from the longitudinal axis A3. In some embodiments of the present disclosure, when the second threshold is exceeded, a thread of the mating connector 20 can skip over or pass a thread of the coupling device 200, thereby reducing the resistance to separation of the coupling device 200 from the mating connector 20.

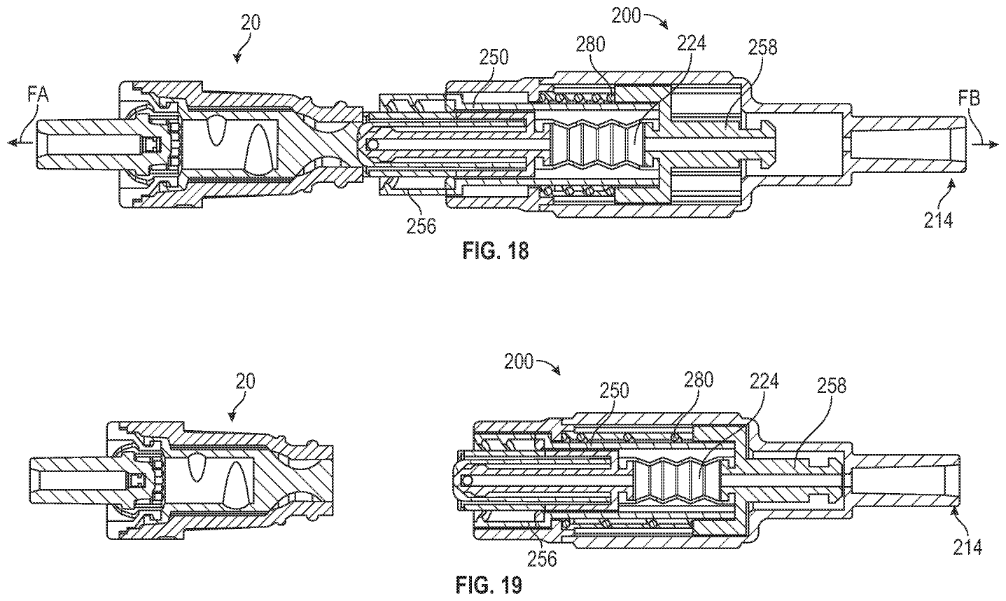

When the mating connector 20 separates from the coupling device 200, the valve 270 can move from the open position to the closed position such that the passage 259 is obstructed and movement of a fluid therethrough is resisted, as shown in FIG. 18. In some embodiments, the bellows 224 expands to exert a force on the valve 270 to move the valve from the open position to the closed position.

After the pulling or tension force FA, FB is reduced to less than the first threshold, the inner body 250 can move from the second position to the first position so that the coupling device 200 is in the first configuration as shown in FIG. 19. The inner body 250 can move to the first position so that the coupling device 200 is in the first configuration when the pulling the tension force FA, FB is reduced or when the mating connector 20 is separated from the coupling device 200 such that the pulling the tension force FA, FB is no longer present.

Movement of the inner body 250 from the second position to the first position is caused by the inner body biasing element 280 expanding to exert a force against the first end portion 254 of the inner body 250 in a direction toward the first end 214 of the housing.

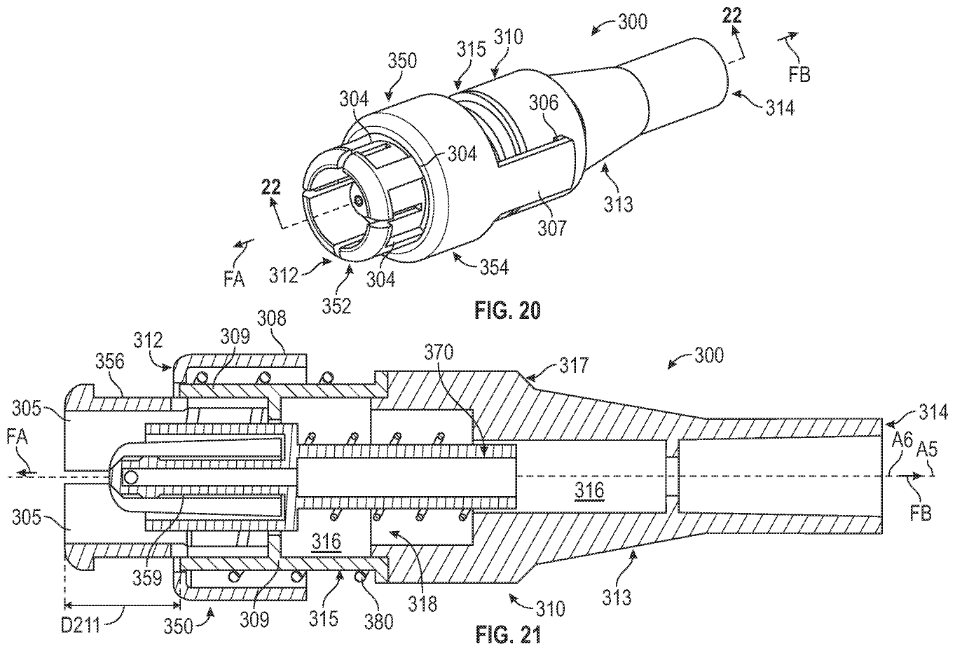

In some embodiments of present disclosure, a coupling device 300 can have a movable cuff configured to couple with the mating connector 20. The coupling device 300 is shown in FIGS. 20-24, and illustrates the coupling device 300 having a housing 310 and cuff 350 that is movable, relative to the housing 310.

The cuff 350 includes a portion forming a coupling sleeve 356 configured to couple with the mating connector 20. The coupling sleeve can include any structure configured to couple with a mating connector 20, such as a thread, a barb, or an interference fit between a surface of the coupling sleeve 356 and the mating connector 20.

The coupling device 300 has a first configuration in which the cuff 350 is in a first position relative to the housing 310, whereby the coupling sleeve 356 of the cuff can resist separation or disconnection of the mating connector 20 from the coupling device 300.

When the cuff 350 is in the first position, a distal end of the cuff is approximately aligned or coplanar with an end of the housing. In some embodiments of the present disclosure, when the cuff 350 is in the first position, the coupling sleeve 356 of the cuff is approximately aligned or coplanar with a support column formed by the housing. In some aspects of the present disclosure, there is a distance D221 between a distal end of the coupling sleeve 356 and a distal end of the support column of the housing.

The coupling device 300 also has a second configuration in which the cuff 350 is moved, relative to the housing 310, to a second position. In the second configuration, the coupling sleeve 356 of the cuff can permit separation or disconnection of the mating connector 20 from the coupling device 300.

To permit separation or disconnection of the mating connector 20 from the coupling device 300, the cuff 350 is oriented so that the coupling sleeve 356 extends distal to the end of the housing. In some aspects of the present disclosure, there is a distance D211 between a distal end of the coupling sleeve 356 and a distal end of the support column of the housing 310, where the distance D211 is greater than the distance D221.

Although there may be a distance D221 between the distal end of the coupling sleeve 356 and the distal end of the support column of the housing, it should be understood that the present disclosure contemplates that the distance D221 can be zero such that the distal end of the end of the coupling sleeve 356 is coplanar with the distal end of the support column of the housing.

When the coupling device 300 is in the first configuration, with the cuff 350 in the first position, the coupling device 300 can resist unintended separation of the mating connector 20 from the coupling device 300 by resisting biasing or flexing of the coupling sleeve 356 away from mating connector 20. In some embodiments of the present disclosure, biasing of the coupling sleeve radially outward is resisted by the housing 310, such that, when the mating connector 20 and the coupling device 300 are pulled away from each other, the sleeve will engage against a portion of the housing 310, thereby resisting movement of the mating connector 20 out of the sleeve.

The coupling device 300 can be moved from the first configuration to the second configuration, when a pulling or tension force exceeds a threshold of the coupling device 300. For example, when a patient moves in an unintended manner, or when the medical tubing is pulled away from the patient, a pulling or tension force FA, FB may be applied to the mating connector 20 or the tubing 16 in a direction away from the coupling device 300. In at least some instances of the present disclosure, a pulling or tension force FA is in a distal direction relative to the coupling device, and a pulling or tension force FB is in a proximal direction.

If the pulling or tension force FA, FB exceeds a first threshold of the coupling device 300, any of the housing 310 and the cuff 350 can move away from each other so that the cuff 350 moves from the first position to the second position. The movement of the housing 310 and the cuff 350, relative to each other, can be along a longitudinal axis A5 of the cuff 350.

If a pulling or tension force FA, FB exceeds a second threshold of the coupling device 300, the coupling sleeve of the cuff 350 is configured such that the engagement of the mating connector 20 against the coupling sleeve causes at least a portion of the coupling sleeve to bias or flex away from the mating connector 20. When the coupling sleeve flexes away from the mating connector 20, the resistance to separation of the coupling device 300 from the mating connector 20 is reduced, relative to when the coupling device 300 is in the first configuration, thereby permitting the coupling device 300 to separate from the mating connector 20. In some embodiments, engagement of a thread of the mating connector 20 against a thread of the cuff 350 when the force FA, FB exceeds the second threshold causes the portion of the sleeve to flex away from the mating connector 20. In some aspects of the present disclosure, a thread of the coupling device 300 can skip over or pass a thread of the mating connector 20 when the force FA, FB exceeds a second threshold of the coupling device 300.

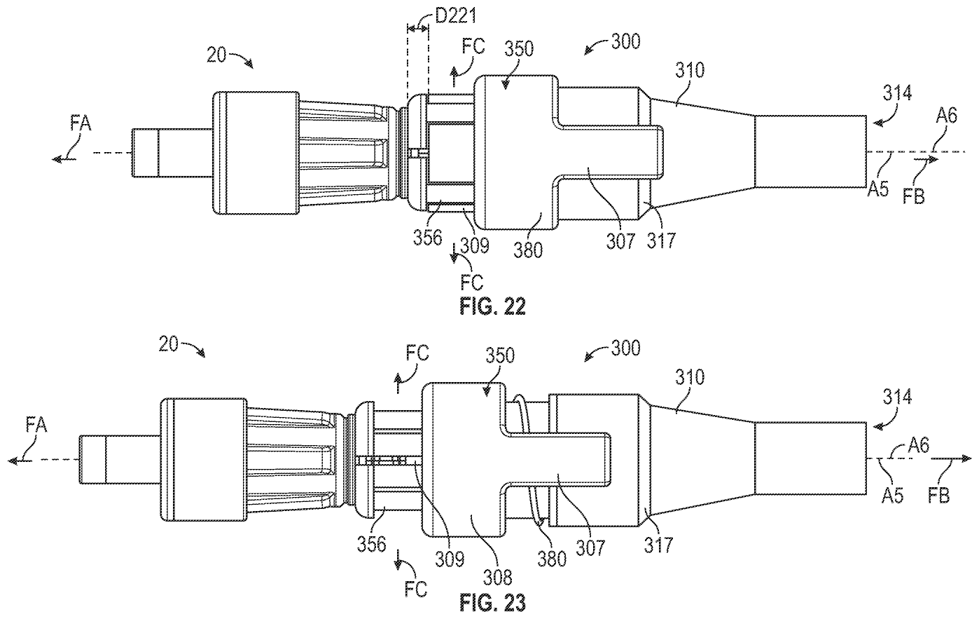

FIG. 21 illustrates a cross-sectional elevation view of the coupling device 300 in the second configuration with the cuff 350 in the second position. The coupling device 300 includes a housing 310, a cuff 350, and a cuff biasing member 380. The cuff 350 is configured to resist and/or permit separation of a mating connector 20 from the coupling device. In some aspects of the present disclosure, separation of a mating connector 20 from the coupling device is resisted when the cuff is in a first position, and separation of a mating connector 20 from the coupling device is permitted when the cuff is in a second position. The first position of the cuff 350 is shown, for example, in FIG. 22, and the second position of the inner body 150 is shown, for example, in FIG. 23.

The housing 310 includes a proximal end portion 313 forming a first end 314, a distal end portion 315 forming a second end 312. The first end 314 is opposite to the second end 312, and a longitudinal axis A6 is defined between the first and second ends. An inner surface 316 of the housing forms a cavity 318 that extends between the first end 314 and the second end 312 of the housing and is configured to receive a portion of the cuff 350 therein.

The distal end portion 315 of the housing forms a support column 309 and the luer protrusion 357. The support column 309 extends axially along and is spaced apart from the luer protrusion 357. In some embodiments of the present disclosure, a plurality of support columns can be positioned around circumference of the luer protrusion 357, such that a distal end of each support column 309 of the plurality of support columns is flexible toward or away from the luer protrusion 357.

Each support column 309 of the plurality of support columns can be spaced apart from an adjacent support column 309 by a distance. The space between adjacent support columns 309 can permit the distal portion of each support column 309 to be biased relative to the longitudinal axis A6 of the housing. Additionally, the space between adjacent support columns 309 can permit a portion of the cuff 350 to be positioned and moveable therebetween.

The space between adjacent support columns 309 can be formed by a channel 358 that extends through the housing 310, in a direction from the second end 312 toward the first end 314 of the housing.

The luer protrusion 357 is positioned radially ward from the support columns 309, and is configured to engage against a mating connector 20 and form a portion of a fluid passageway through the coupling device 300.

The luer protrusion 357 is formed by the distal end portion 315 of the housing and can have an outer surface forming a male luer. The structure and orientation of the luer protrusion 357 is such that the luer protrusion extends through the recess defined by the support columns 309 which extends around a perimeter or circumference of the luer protrusion 357.

The support columns 309 have an inner surface that is spaced apart from the outer surface of the luer protrusion 357, where the space between the support columns 309 and the luer protrusion 357 is configured to receive a portion of mating connector 20 therebetween.

The inner surface of the luer protrusion 357 forms a portion of the valve passage 359 through the luer protrusion 357. The valve passage 359 can permit a fluid to through the coupling device 300 when coupled with a mating connector 20.

To couple with a mating connector 20, the coupling device 300 includes a cuff 350 at the distal end portion 315 thereof. The cuff 350 has a proximal portion 354 and a distal end portion 352, where the proximal portion 354 forms a retention sleeve 308, and the distal end portion 352 forms a coupling sleeve 356.

The retention sleeve 308 is formed by the proximal portion 354 of the cuff, and is configured to extend around an outer surface of the housing 310. When coupled with the housing 310, the retention sleeve 308 is axially aligned with the distal end portion 315 of the housing and extends around the support columns 309.

In some embodiments of the present disclosure, the retention sleeve 308 comprises a retention tab 307 that is configured to engage against the housing 310 to resist longitudinal movement of the cuff 350.

The retention tab 307 extends from the retention sleeve 308 toward the first end 314 of the housing when the cuff 350 is coupled to the housing. When the cuff 350 is in the first position, the retention tab 307 engages against a portion of the housing 310 to resist movement of the cuff 350.

The retention tab 307 is configured to engage against the housing 310 to resist longitudinal movement of the cuff in a direction away from the proximal end portion 313 of the housing. When a pulling or tension force FA, FB on the coupling device 300 exceeds a first threshold, the retention tab 307 can disengage from the housing 310 to permit the cuff 350 to move in a direction away from the proximal end portion 313 of the housing, from the first position to the second position.

The retention tab 307 can disengage from the housing 310 when a portion of the retention tab 307 flexes or biases. In some embodiments, a distal portion of the retention tab 307 is configured to bias in a direction away from the housing 310 when the pulling or tension force FA, FB on the coupling device 300 exceeds the first threshold.

Further, in some embodiments of the present disclosure, the outer surface of the housing 310 forms a ridge 317 configured to be engaged by the retention tab 307. The ridge 317 can be formed by a portion of the outer surface of the housing 310 that is a concave or convex surface, including any of a dimple, channel, protrusion, and/or wall.

Referring to FIG. 21, in some embodiments, the ridge 317 is formed between the proximal end portion 313 and the distal end portion 315 of the housing. The ridge 317 between the proximal and distal end portions 313, 315 of the housing can be formed by the proximal end portion 313 having a cross-sectional width that is less than across-sectional width of the distal end portion 315.

In some embodiments of the present disclosure, the retention tab 307 comprises a protrusion 306 that is configured to engage against the housing 310 to resist longitudinal movement of the cuff 350. The protrusion 306 can extend in a direction toward the housing 310, and can be configured to engage against the ridge 317 when the cuff is in the first position.

Although the retention tab 307 is configured to resist longitudinal movement of the cuff 350, it should be understood that the present disclosure contemplates embodiments in which the retention tab 307, or another portion of the cuff 350, is also configured to resist rotation of the cuff 350 relative to the housing 310. Resisting rotation of the cuff 350, relative to the housing 310, can permit the cuff 350 to be coupled to a mating connector 20, such as by engaging a thread of the coupling sleeve 356 with a thread of the mating connector 20.

The coupling sleeve 356 can have a discontinuous surface or perimeter to permit the coupling sleeve to flex or bias toward or away from the longitudinal axis A5. The discontinuous surface can be formed by a channel that extends through a distal-most end of the distal end portion 352 of the cuff in a direction toward the retention sleeve 308.

In some embodiments of the present disclosure, two or more channels extend through the coupling sleeve 356 to form a plurality of flexible fingers 305, wherein each finger is defined between adjacent channels. As such, the coupling sleeve 356 can include a first flexible finger 305 and a second flexible finger 305. In some aspects of the present disclosure, the coupling sleeve 356 includes four flexible fingers 305, which are separated from each other by three channels through the coupling sleeve 356.

The coupling sleeve 356 and the retention sleeve 308 are joined together by a portion of the cuff 350 forming a bridge 304. The bridge 304 extends from the retention sleeve 308 toward the coupling sleeve 356, in a direction that is radially inward toward the longitudinal axis A5. Because the bridge 304 extends radially inward, a cross-sectional width defined by the inner surface of the coupling sleeve 356 is less than a cross-sectional width defined by the inner surface of the retention sleeve 308.

In some embodiments of the present disclosure, a plurality of bridges 304 extend between the coupling sleeve 356 and the retention sleeve 308, thereby forming a space or passage configured to receive a support columns 309 therethrough. The passage for the support column 309 can be bounded by the bridge 304, the coupling sleeve 356, and the retention sleeve 308. In some aspects of the present disclosure, the bridge 304 is aligned with the flexible fingers 305 such that the passage between each bridge 304 is aligned with a channel through the coupling sleeve 356.

To bias the cuff 350 toward the second position, the coupling device 300 can include a cuff biasing member 380 between the housing 310 and the cuff 350. The cuff biasing member 380 is positioned to helically extend around the outer surface of the housing 310. In some embodiments of the present disclosure, the cuff biasing member 380 extends along the distal end portion 315 of the housing, between the support columns 309 and the inner surface of the retention sleeve 308.

The cuff biasing member 380 is configures such that in the second position of the cuff 350, the cuff biasing member 380 is in an expanded or neutral state, and in the first position of the cuff 350, the cuff biasing member 380 is compressed. The cuff biasing member 380 is maintained in the compressed orientation with the cuff 350 in the first position by engagement of the retention tab 307 against the housing 310.

To permit or resist a fluid flow through the luer protrusion, the coupling device 300 can include a valve 370. The valve 370 includes features corresponding to the valve 170 of the coupling device 100, excepts where disclosed herein.

The valve 370 is positioned in the cavity of the housing 310, and is movable between the first end 314 and the second end 312 of the housing. The valve 370 can also move relative to the housing 310 and to the cuff 350, such that the valve 370 is movable when the cuff 350 is in any of the first and second positions.

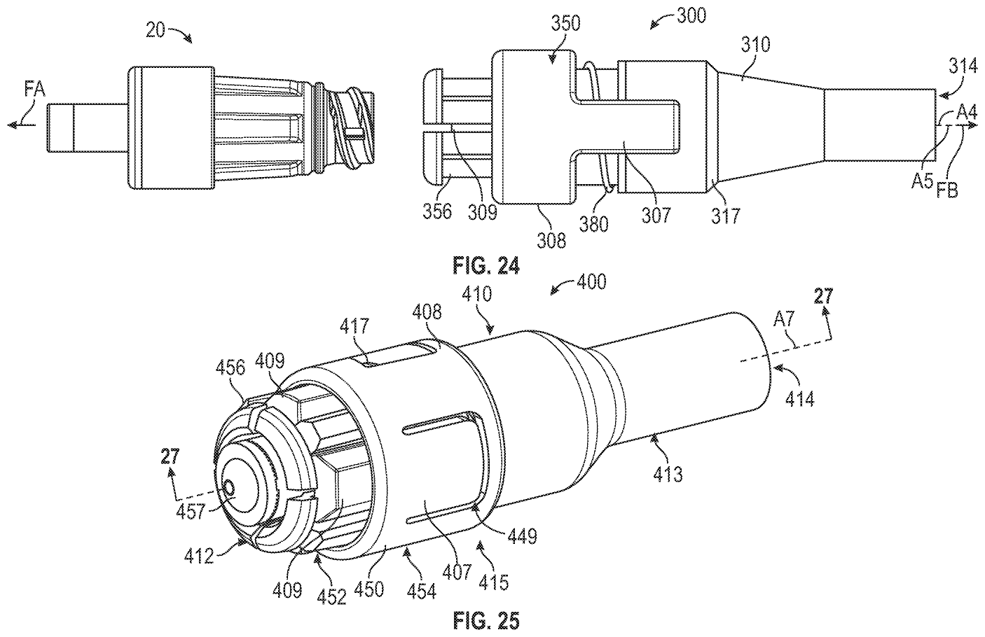

The coupling device 300 and a mating connector 20 are shown together in FIGS. 22-24, FIG. 22 shows the coupling device 300 is in the first configuration, FIG. 23 shows the coupling device 300 in the second configuration, and FIG. 24 shows the coupling device 300 moving from the second configuration to the first configuration after separation of the mating connector 20 from the coupling device 300.

In the first configuration of the coupling device 300, the cuff 350 is in the first position relative to the housing 310, whereby the coupling sleeve 356 extends along the support column 309. Engagement of the coupling sleeve 356 with the mating connector 20 resists separation of the mating connector 20 from the coupling device 300 when a pulling or tension force FA, FB is exerted on the mating connector 20 and/or the coupling device 300. Additionally, in the first configuration of the coupling device 300, the valve 370 is the open position such that an unobstructed fluid pathway through the coupling device 300 and the mating connector 20 is formed.

Separation of the mating connector 20 from the coupling device 300 is resisted when the coupling device 300 is in the first configuration by resisting movement of the support column 309 radially outward. In some embodiments of the present disclosure, movement of the support column 309 by biasing or flexing radially outward is resisted by the position of the coupling sleeve 356 extending along the support column 309. In particular, biasing or flexing of the support column 309 radially outward in a direction FC is resisted by engagement of the support column 309 against the coupling sleeve 356.

The coupling device 300 is moved to the second configuration after a pulling or tension force FA, FB exceeds a first threshold of the coupling device 300. When the mating connector 20 is coupled with the coupling device 300, and a pulling or tension force exceeds a threshold of the coupling device 300, the mating connector 20 moves with the cuff 350 relative to the housing 310. As the coupling device 300 is moved to the second configuration, the cuff 350 is moved in a direction away from the first end 314 of the housing, as shown in FIG. 23.

In some embodiments of the present disclosure, when the coupling device 300 moves from the first configuration to the second configuration, movement of the cuff 350 and the mating connector 20 in a direction away from the first end 314 of the housing permits the valve 370 to move from the open position to the closed position. In some embodiments of the present disclosure, the valve 370 remains in the open position when the coupling device 300 is in the second configuration.

When the pulling or tension force FA, FB exceeds a second threshold of the coupling device 300, the engagement of the mating connector 20 against the cuff 350 causes at least a portion of the coupling sleeve 356 to bias or flex in a direction FC away from the longitudinal axis A5. In some embodiments of the present disclosure, when the second threshold is exceeded, a thread of the mating connector 20 can skip over or pass a thread of the coupling device 300, thereby reducing the resistance to separation of the coupling device 300 from the mating connector 20.

When the mating connector 20 separates from the coupling device 300, as shown in FIG. 24, the valve 370 is in the closed position such that the fluid pathway though the coupling device 300 is obstructed and movement of a fluid therethrough is resisted.

In some embodiments of the present disclosure, the coupling device 300 and the mating connector 20 can be reconnected or coupled together after separation such that the fluid pathway through the coupling device 300 and the mating connector 20 is unobstructed.

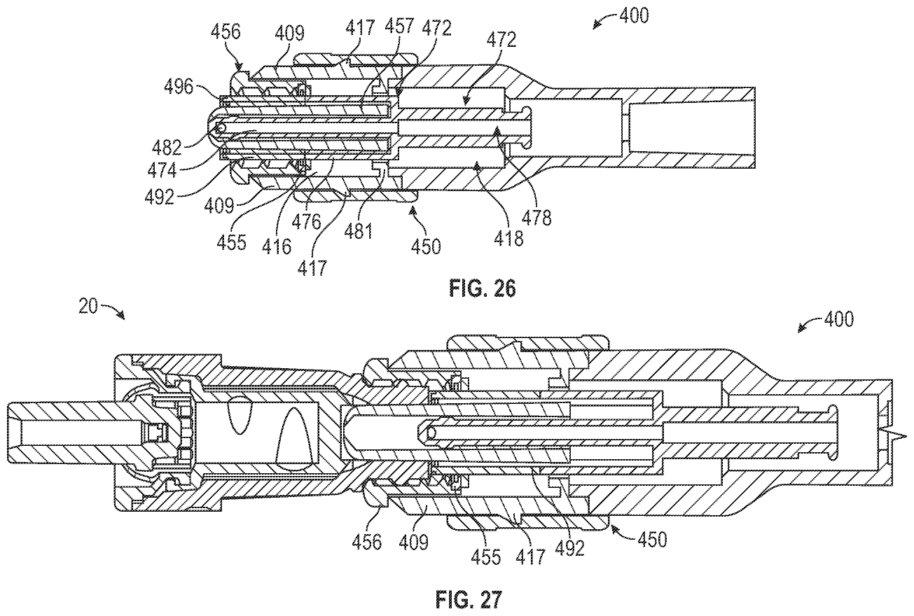

FIGS. 25-33 illustrate another embodiment of a coupling device 400 having features of the present disclosure, including a cuff 450 that is movable relative to the housing 410. The coupling device 400 can include features disclosed elsewhere in the present disclosure and shown in one or more embodiment, and as such, like features already described are not repeated herein for clarity and brevity.

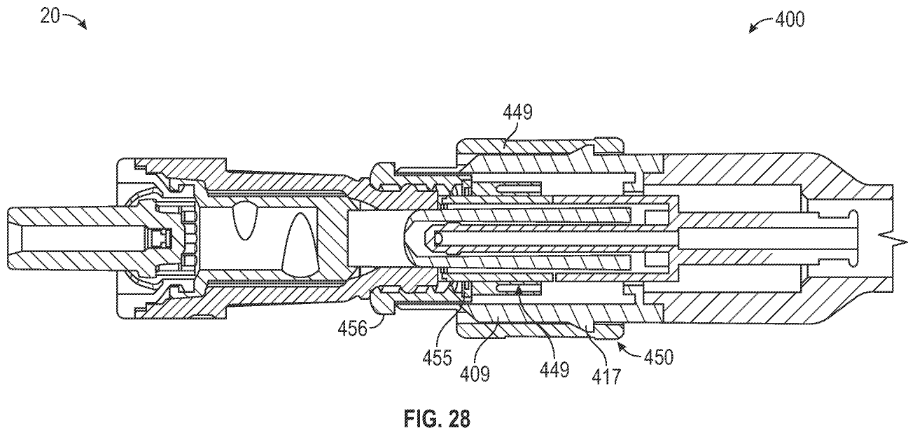

The coupling device 400 includes a cuff 450 having features like the cuff 350 disclosed with reference to the coupling device 300, where the cuff 350 is configured to resist unintended separation of the mating connector 20 from the coupling device 400 by resisting biasing or flexing of a coupling sleeve away from mating connector 20 when the coupling device 400 is in a first configuration. The first configuration of the coupling device 400 is shown, for example, in FIG. 27, where the cuff 450 is in a first position to resist separation of the mating connector 20 from the coupling device 400. In a second configuration of the coupling device 400, as shown, for example, in FIG. 28, the cuff is in a second position to permit separation of the mating connector 20 from the coupling device 400.

The coupling device 400 includes a housing 410 and a cuff 450, the housing 410 has a proximal end portion 413 forming a first end 414, a distal end portion 415 forming a second end 412. A longitudinal axis A7 is defined between the first and second ends of the housing.

The distal end portion 415 of the housing forms support columns 409 and a luer protrusion 457 positioned radially inward from the support columns 409.

The support columns 409 have an inner surface that is spaced apart from the outer surface of the luer protrusion 457, where the space between the support columns 409 and the luer protrusion 457 is configured to receive portions of a mating connector 20 and the cuff 450 therebetween. Additionally, a distal end of each support column 409 is flexible toward or away from the luer protrusion 457.

Each of the support columns 409 are spaced apart from each other by a distance. The space between adjacent support columns 409 can permit the distal portion of each support column 409 to be flexible relative to an adjacent support column 409. In use, the support columns 409 can be flexed or biased relative to the longitudinal axis A7 of the housing. Additionally, the space between adjacent support columns 409 can permit a portion of the cuff 450 to be positioned and moveable therebetween.

An inner surface 416 of the housing forms a cavity 418 that extends between the first end 414 and the second end 412 of the housing and is configured to receive at least a portion of the cuff 450 therein.

An outer surface of the housing can include a ridge 417 extends radially outward in a direction away from the longitudinal axis A7. The ridge 417 is configured to engage against the cuff 450 to resist or limit any of a longitudinal and a rotational movement of the cuff 450. However, in some embodiments of the present disclosure, any of the housing 410 and the cuff 450 can have a ridge configured to engage against the other of the housing 410 and the cuff 450. In some aspect so the present disclosure, any of the housing 410 and the cuff 450 include a channel or concave surface configured to receive the ridge therein.

The cuff 450 is positioned at the distal end portion 415 of the coupling device 400, and includes a proximal portion 454 and a distal end portion 452. The proximal portion 454 forms a retention sleeve 408 configured to couple with the housing 410, and the distal end portion 452 forms a coupling sleeve 456 configured to couple with a mating device 20.

The cuff 450 is coupled with the housing 410 such that the retention sleeve 408 extends along to the outer surface of the housing 410 and the coupling sleeve 456 extends along the inner surface 416 of the housing. The cuff 450 is oriented relative to the housing 410 such that the support columns 409 extends between the retention sleeve 408 and the coupling sleeve 456, and the cuff 450 is movable in a longitudinal direction relative to the housing 410.

To resist or limit movement of the cuff 450, relative to the housing 410, the retention sleeve 408 comprises a retention tab 407 configured to engage against the housing 410. The retention tab 407 is configured to engage against a portion of the housing 410 to resist movement of the cuff 450 from the first position to the second position. However, when a pulling or tension force FA, FB on the coupling device 400 exceeds a first threshold, the retention tab 407 can disengage from the portion of the housing 410 to permit the cuff 450 to move from a first position to a second position.

In some embodiments of the present disclosure, the outer surface of the housing 410 forms a recess 446 configured to be engaged by the retention tab 407 to resist movement of the cuff 450 when the cuff 450 is in the first position. The recess 446, which is shown in FIGS. 32 and 34, can be formed by a concave portion of the housing outer surface, such as a channel, groove, or cut-out; however, it should also be understood that the present disclosure contemplates embodiments in which the outer surface of the housing 410 can have a convex surface forming any of a protrusion or ridge configured to engage against the retention tab 407.

Disengagement of the retention tab 407 from the housing 410 can result from a portion of the retention tab 407 configured to flex or bias. In some embodiments, a distal portion of the retention tab 407 is configured to bias in a direction away from the housing 410 when the pulling or tension force FA, FB on the coupling device 400 exceeds the first threshold.

The proximal portion 454 of the cuff can include windows 449 forming openings extending through the inner and outer surfaces of the cuff 450. The windows 449 can include the retention tab 407 positioned therein and/or can be configured to receive a portion of the housing 410, such as the ridge 417, therein.

In some examples of the present disclosure, each of a first plurality of windows can be configured with a retention tab 407 therein, and each of a plurality of second windows can be positioned between adjacent windows of the first plurality of windows, and can be configured to receive the ridge 417 of the housing therein.

Although in some embodiments of the present disclosure, the distal portion of the retention tab 407 extends in a direction from the distal portion 452 toward the proximal portion 454 of the cuff, it should be understood that the present disclosure contemplates embodiment in which the retention tab 407 extends from the proximal portion 454 toward the distal portion 452 of the cuff, or can extend in a direction that is transverse, relative to the longitudinal axis A7.

The ridge 417 extends into the window 449 to resist unintended longitudinal and rotational movement of the cuff 450. To resist unintended longitudinal and rotational movement of the cuff 450, the ridge 417 can be configured to be positioned within a distal portion of the window when the cuff 450 is in the first positioned, and within a proximal portion of the window when the cuff 450 is in the second position.

At the distal end portion 452 of the cuff, the coupling sleeve 456 comprises features like the coupling sleeve 356 of the cuff for the coupling device 300, including a discontinuous circumference configured to permit the coupling sleeve to flex or bias toward or away from the longitudinal axis A7. The discontinuous surface can be formed by one or more channel that extends through a distal-most end of the distal end portion 452 of the cuff in a direction toward the retention sleeve 408.

The cuff 450 is coupled to the housing 410 with the retention sleeve 408 extending along the outer surface of the support columns 409, the coupling sleeve 456 extending along the inner surface of the support columns 409, and a portion of a valve 470 positioned between the inner surface of the coupling sleeve 456 and the outer surface of the luer protrusion 457.

The valve 470 includes features like the valve 170 of the coupling device 100, including a proximal end portion 471 and a distal end portion 472, where the distal end portion forms forming a post 474 and an arm 476. The inner surface of the valve 470 defines a fluid channel 478 extending through the proximal and distal end portions of the valve 470. The fluid channel 478 extends through an opening 182 formed at the distal end portion 472 of the post and extending radially between the inner and outer surfaces of the post 474.

A cylindrical extension 492 of the arm is positioned between the inner surface of the coupling sleeve 456 and the outer surface of the luer protrusion 457. To form a seal between the valve 470 and the luer protrusion 457, a valve seal 496 extends from any of the arm 476 and the cylindrical extension 492, and is configured to engage against the outer surface of the luer protrusion 457.

To form a seal between the cuff 450 and the valve 470, the coupling sleeve 456 comprises a cuff seal 455 that extends between the inner surface of the coupling sleeve 456 and the cylindrical extension 492. As the valve 470 moved between the open and closed positions, as shown in FIGS. 27 and 28, respectively, the valve seal 496 and the cuff seal 455 can resist unintended materials, such as fluids and/or solids, from moving into or out of the cavity of the housing 110.

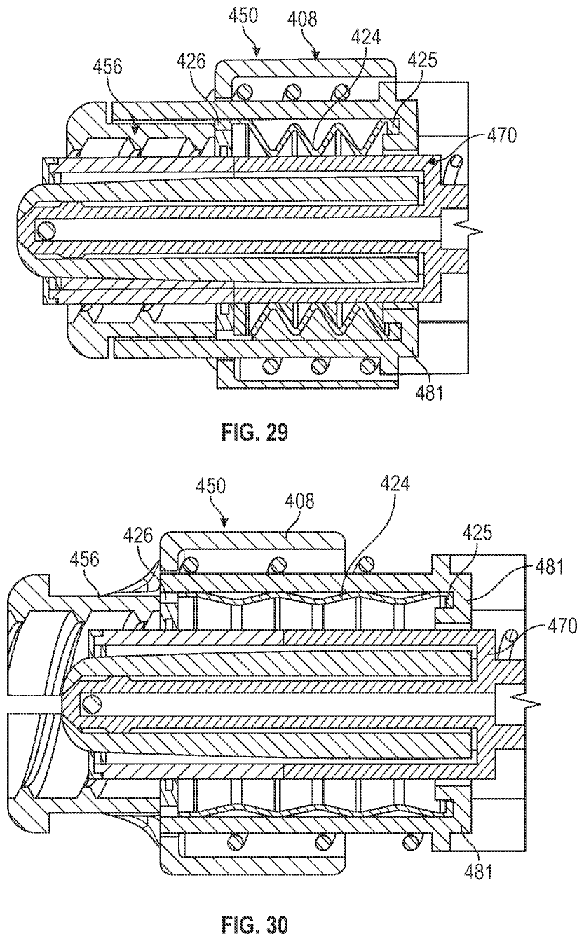

In some embodiments of the present disclosure, a coupling device can include a barrier sleeve 424 extending between the housing 410 and the cuff 450, as shown in FIGS. 29 and 30. The barrier sleeve 424 has a first end 425 engaged against a portion of the housing that extends radially inward toward the valve 470, and a second end 426 engaged against the coupling sleeve 456.

The portion of the housing that extends radially inward toward the valve 470 can be formed by a flange 481 that extends inward from the inner surface 416 of the housing. To maintain a seal or barrier, the first and second ends 525, 426 of the barrier sleeve remain engaged against the housing 410 and cuff 450, respectively, when the cuff 450 is in the first and second positions. As such, the barrier sleeve 424 comprises a length between the first and second ends 525, 426 that is greater than the length between the flange 481 and the coupling sleeve 456 when the cuff 450 is in the first position.

When the cuff 450 moves from the first position to the second position, the barrier sleeve 424 can compensate for the movement of the cuff 450 by expanding or stretching linearly. To expanding or stretching linearly, the barrier sleeve 424 can be formed as a bellows.

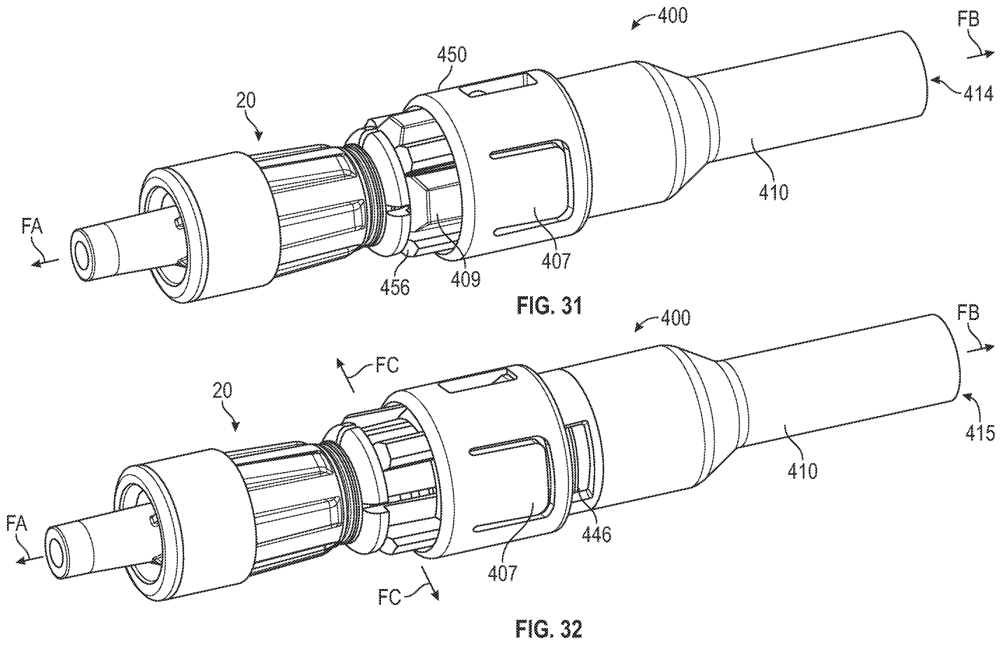

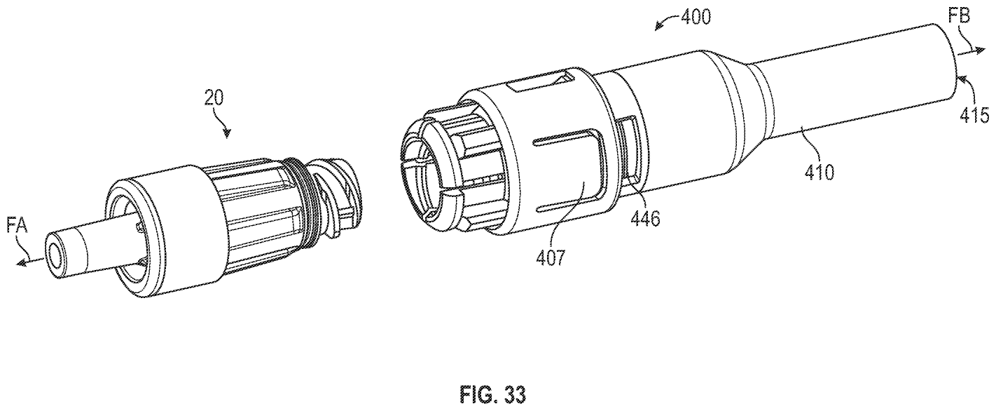

The coupling device 400 and a mating connector 20 are shown together in FIGS. 31-33, FIG. 32 shows the coupling device 400 is in the first configuration, FIG. 32 shows the coupling device 400 in the second configuration with the mating connector 20 attached thereto, and FIG. 33 shows the coupling device 400 after separation of the mating connector 20 from the coupling device.

In the first configuration of the coupling device 400, the cuff 450 is in the first position relative to the housing 410, whereby the coupling sleeve 456 extends along the support column 409. Engagement of the coupling sleeve 456 with the mating connector 20 resists separation of the mating connector 20 from the coupling device 400 when a pulling or tension force FA, FB is exerted on the mating connector 20 and/or the coupling device 400. Additionally, in the first configuration of the coupling device 400, the valve 470 is the open position such that an unobstructed fluid pathway through the coupling device 400 and the mating connector 20 is formed.

Separation of the mating connector 20 from the coupling device 400 is resisted when the coupling device 400 is in the first configuration by resisting movement of the support column 409 radially outward. In some embodiments of the present disclosure, movement of the support column 409 by biasing or flexing radially outward is resisted by the position of the coupling sleeve 456 extending along the support column 409. In particular, biasing or flexing of the support column 409 radially outward in a direction FC is resisted by engagement of the support column 409 against the coupling sleeve 456.

The coupling device 400 is moved to the second configuration after a pulling or tension force FA, FB exceeds a first threshold of the coupling device 400. When the mating connector 20 is coupled with the coupling device 400, and a pulling or tension force exceeds a threshold of the coupling device 400, the mating connector 20 moves with the cuff 450 relative to the housing 410. As the coupling device 400 is moved to the second configuration, the cuff 450 is moved in a direction away from the first end 414 of the housing, as shown in FIG. 32.

When the pulling or tension force FA, FB exceeds a second threshold of the coupling device 400, the engagement of the mating connector 20 against the cuff 450 causes at least a portion of the coupling sleeve 456 to bias or flex in a direction FC away from the longitudinal axis. In some embodiments of the present disclosure, when the second threshold is exceeded, a thread of the mating connector 20 can skip over or pass a thread of the coupling device 400, thereby reducing the resistance to separation of the coupling device 400 from the mating connector 20.

When the mating connector 20 separates from the coupling device 400, as shown in FIG. 33, the valve 470 is in the closed position such that the fluid pathway though the coupling device 400 is obstructed and movement of a fluid therethrough is resisted. To re-establish the fluid pathway, the mating connector 20 and the coupling device 400 can be coupled together once again, wherein coupling the mating connector 20 to the coupling device 400 would cause the coupling device to once again be in the first configuration.

Accordingly, the present disclosure provides features of coupling devices that can provide for efficient and safe maintenance of fluid connections, such as the connections used for transferring medical fluids toward or away from a patient. Further, the coupling devices of the present disclosure can maintain the fluid connection by resisting unintended disconnection when a pulling or tension force is applied to the coupling device, while also permitting disconnection of the fluid connection when a pulling or tension force exceeds a threshold, thereby preventing injury to a patient or a caregiver. Additionally, the coupling devices of the present disclosure permit efficient and safe reconnection of the fluid line by permitting the coupling device to be reconnected to the fluid transfer device, thereby reestablishing the fluid pathway without requiring replacement of the coupling device.

Accordingly, the coupling device 100 can provide for efficient and safe maintenance of fluid connections, can maintain the fluid connection of a medical tubing, and can permit disconnection and reconnection of the fluid pathway with a patient.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1. A coupling device for medical tubing, the coupling device comprising: a housing having an inner surface forming a cavity, a first opening into the cavity, and a second opening into the cavity; and an inner body having a fluid passage, extending through the inner body, and a coupling sleeve, wherein the inner body is positioned within the cavity of the housing with the coupling sleeve extending toward the first opening of the housing such that the housing and the inner body are movable relative to each other; wherein, in a first position of the inner body, biasing of at least a portion of the coupling sleeve in a direction that is radially outward is resisted by engagement of the coupling sleeve with the housing; and wherein, in a second position of the inner body, the coupling sleeve is moved through the first opening in a direction that is away from the second opening of the housing such that the at least a portion of the coupling sleeve can be biased radially outward.

Clause 2. The coupling device of Clause 1, wherein the inner body comprises at least one channel that extends through the coupling sleeve in a direction from a first end of the inner body toward a second end of the inner body.

Clause 3. The coupling device of Clause 2, wherein the at least one channel comprises a first channel and a second channel, wherein the second channel is spaced apart from the first channel, and where a first portion of the coupling sleeve between the first and second channels forms a first flexible finger, and a second portion of the coupling sleeve between the first and second channels forms a second flexible finger.

Clause 4. The coupling device of any one of Clauses 1 to 3, wherein the inner body comprises a luer tip that extends within the coupling sleeve, toward a first end of the inner body.

Clause 5. The coupling device of any one of Clauses 1 to 4, further comprising a biasing element positioned between the first end of the housing and the inner body.

Clause 6. The coupling device of any one of Clauses 1 to 5, wherein the inner body comprises a spline that extends from an outer surface of the inner body, in a direction that is radially outward away from an outer surface of the inner body.

Clause 7. The coupling device of Clause 6, wherein the spline is configured to be positioned within a groove extending along the inner surface of the housing such that the housing and the inner body are rotationally coupled.

Clause 8. The coupling device of Clause 7, wherein the housing and the inner body are movable, relative to each other, along a length of the groove.

Clause 9. A coupling device for medical tubing, the coupling device comprising: a housing having a first end, a second end, and an inner surface forming a cavity, wherein the first end comprises a first opening into the cavity, and the second end comprises a second opening into the cavity; an inner body having a first end portion, a second end portion, a fluid passage extending through the first and second end portions of the inner body, the first end portion comprising a coupling sleeve formed by a wall having an inner surface that extends around a longitudinal axis defined by the fluid passage to form a recess extending from a first end of the inner body toward the second end portion, wherein the inner body is positioned within the cavity of the housing such that the housing and the inner body are movable relative to each other; and a biasing element positioned between the first end of the housing and the inner body; wherein, in a first position of the inner body, the first end of the inner body is longitudinally aligned with the first end of the housing such that biasing of at least a portion of the wall away from the longitudinal axis is resisted by the housing, and in a second position of the inner body, the second end of the housing is moved away from the second end portion of the inner body such that the first end of the inner body is longitudinally spaced apart from the first end of the housing and the at least a portion of the wall can be biased away from the longitudinal axis.

Clause 10. The coupling device of Clause 9, wherein the inner body comprises at least one channel that extends through the wall, from the first end of the inner body toward the second end portion of the inner body.

Clause 11. The coupling device of Clause 10, wherein the at least one channel comprises a first channel and a second channel, wherein the second channel is spaced apart from the first channel, and where a first portion of the wall between the first and second channels forms a first flexible finger, and a second portion of the wall between the first and second channels forms a second flexible finger.

Clause 12. The coupling device of any one of Clauses 9 to 11, wherein the inner body comprises a thread that extends along the inner surface of the wall.

Clause 13. The coupling device of any one of Clauses 9 to 12, wherein the inner body comprises a luer tip that extends within the coupling sleeve, from a bottom surface of the recess toward the first end of the inner body.

Clause 14. The coupling device of any one of Clauses 9 to 13, wherein the first opening of the housing comprises a first cross-sectional width, and an outer surface of the wall of the inner body comprises a second cross-sectional width, and wherein the first cross-sectional width is equal to or greater than the second cross-sectional width, such that, the first end portion of the inner body is longitudinally movable between the first and second positions through the first opening.

Clause 15. The coupling device of any one of Clauses 9 to 14, wherein the inner body comprises a spline that extends from an outer surface of the inner body, in a direction that is radially outward away from the longitudinal axis.

Clause 16. The coupling device of Clause 15, wherein the spline is configured to be positioned within a groove extending along the inner surface of the housing such that the housing and the inner body are rotationally coupled.

Clause 17. The coupling device of Clause 16, wherein the housing and the inner body are movable, relative to each other, along a length of the groove.

Clause 18. The coupling device of any one of Clauses 9 to 17, wherein the housing comprise a ledge that extends radially inward from the inner surface into the cavity.

Clause 19. The coupling device of Clause 18, wherein, in the first position, a portion of the inner body is engaged against the ledge, thereby resisting movement of inner body toward the second end of the housing.

Clause 20. The coupling device of any one of Clauses 9 to 19, wherein the housing comprises a tubing channel that extends through the second end of the housing and intersects the second opening.

Clause 21. The coupling device of Clause 20, wherein at least a portion of the tubing channel comprises a cross-sectional width that is greater than a cross-sectional width of the second opening.

Clause 22. The coupling device of Clause 21, wherein the cross-sectional width of the tubing channel decreases in a direction toward the second opening.

Clause 23. The coupling device of any one of Clauses 9 to 22, wherein a distance from the first end of the inner body to a second end of the inner body is greater than a distance from the first end of the housing to a distal end of the cavity.

Clause 24. The coupling device of any one of Clauses 9 to 23, further comprising a cap coupled to the first end of the housing, the cap forming at least a portion of the first opening of the housing.

Clause 25. The coupling device of any one of Clauses 9 to 24, wherein the biasing element comprises a spring configured to direct the inner body toward the first position.

Clause 26. A coupling device assembly comprising: a housing having a first end, a second end, and an inner surface forming a cavity, wherein the first end comprises a first opening into the cavity, and the second end comprises a second opening into the cavity; an inner body having a first end portion, a second end portion, a fluid passage extending through the first and second end portions of the inner body, the first end portion comprising a coupling sleeve formed by a wall having an inner surface that extends around a longitudinal axis defined by the fluid passage to form a recess extending from a first end of the inner body toward the second end portion, wherein the inner body is positioned within the cavity of the housing such that the housing and the inner body are movable relative to each other; a biasing element positioned between the first end of the housing and the inner body; and a tubing extending through the second opening of the housing and into the cavity, wherein an end portion of the tubing is coupled to the fluid passage of the inner body, wherein, in a first position of the inner body, the first end of the inner body is longitudinally aligned with the first end of the housing such that biasing of at least a portion of the wall away from the longitudinal axis is resisted by the housing, and in a second position of the inner body, the second end of the housing is moved away from the second end portion of the inner body such that the first end of the inner body is longitudinally spaced apart from the first end of the housing and the at least a portion of the wall can be biased away from the longitudinal axis.

Clause 27. The coupling device of Clause 26, wherein, when the inner body is in the first position, a length of the tubing between a second end of the inner body and a distal end of the cavity, adjacent to the second end of the housing, is longer than a distance between the second end of the inner body and the distal end of the cavity.

Clause 28. A method for providing a coupling device for medical tubing, the method comprising: inserting an inner body into a cavity of a housing such that the housing and the inner body are movable relative to each other, and a coupling sleeve defined by a proximal portion of the inner body extends through a first opening at a first end of the housing; inserting a biasing element into the housing such that the biasing element is positioned between a first end of the housing and the inner body; inserting a tubing through a second opening at a second end of the housing, and coupling the tubing to a fluid passage extending through the inner body; wherein, in a first position of the inner body, a first end of the inner body is longitudinally aligned with the first end of the housing such that biasing of at least a portion of coupling sleeve away from a longitudinal axis of the fluid passage of the inner body is resisted by the housing, and in a second position of the inner body, the second end of the housing is moved away from the second end of the inner body such that the first end of the inner body is longitudinally spaced apart from the first end of the housing and the at least a portion of the coupling sleeve can be biased away from the longitudinal axis.

Clause 29. The method of Clause 28 wherein, when the inner body is in the second position and a mating connector, coupled to the coupling sleeve, is moved in a direction away from the first end of the inner body, engagement of the mating connector with the coupling sleeve directs at least a portion of the coupling sleeve away from the longitudinal axis.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A coupling device for medical tubing, the coupling device comprising:
a housing having an inner surface forming a cavity, a first opening into the cavity, and a second opening into the cavity; and
an inner body having a fluid passage, extending through the inner body, and a coupling sleeve, wherein the inner body is positioned within the cavity of the housing with the coupling sleeve extending toward the first opening of the housing such that the housing and the inner body are movable relative to each other;
wherein, in a first position of the inner body, biasing of at least a portion of the coupling sleeve in a direction that is radially outward is resisted by engagement of the coupling sleeve with the housing; and wherein, in a second position of the inner body, the coupling sleeve is moved through the first opening in a direction that is away from the second opening of the housing such that the at least a portion of the coupling sleeve can be biased radially outward.

2. The coupling device of claim 1, wherein the inner body comprises at least one channel that extends through the coupling sleeve in a direction from a first end of the inner body toward a second end of the inner body.

3. The coupling device of claim 2, wherein the at least one channel comprises a first channel and a second channel, wherein the second channel is spaced apart from the first channel, and where a first portion of the coupling sleeve between the first and second channels forms a first flexible finger, and a second portion of the coupling sleeve between the first and second channels forms a second flexible finger.

4. The coupling device of claim 1, wherein the inner body comprises a luer tip that extends within the coupling sleeve, toward a first end of the inner body.

5. The coupling device of claim 1, further comprising a biasing element positioned between the housing and the inner body.

6. The coupling device of claim 1, wherein the inner body comprises a spline that extends from an outer surface of the inner body, in a direction that is radially outward away from an outer surface of the inner body.

7. The coupling device of claim 6, wherein the spline is configured to be positioned within a groove extending along the inner surface of the housing such that the housing and the inner body are rotationally coupled.

8. The coupling device of claim 7, wherein the housing and the inner body are movable, relative to each other, along a length of the groove.

9. A coupling device for medical tubing, the coupling device comprising:
a housing having a first end, a second end, and an inner surface forming a cavity, wherein the first end comprises a first opening into the cavity, and the second end comprises a second opening into the cavity;
an inner body having a first end portion, a second end portion, a fluid passage extending through the first and second end portions of the inner body, the first end portion comprising a coupling sleeve formed by a wall having an inner surface that extends around a longitudinal axis defined by the fluid passage to form a recess extending from a first end of the inner body toward the second end portion, wherein the inner body is positioned within the cavity of the housing such that the housing and the inner body are movable relative to each other; and
a biasing element positioned between the first end of the housing and the inner body;
wherein, in a first position of the inner body, the first end of the inner body is longitudinally aligned with the first end of the housing such that biasing of at least a portion of the wall away from the longitudinal axis is resisted by the housing, and in a second position of the inner body, the second end of the housing is moved away from the second end portion of the inner body such that the first end of the inner body is longitudinally spaced apart from the first end of the housing and the at least a portion of the wall can be biased away from the longitudinal axis.

10. The coupling device of claim 9, wherein the inner body comprises at least one channel that extends through the wall, from the first end of the inner body toward the second end portion of the inner body.

11. The coupling device of claim 9, wherein the inner body comprises a thread that extends along the inner surface of the wall.

12. The coupling device of claim 9, wherein the inner body comprises a luer tip that extends within the coupling sleeve, from a bottom surface of the recess toward the first end of the inner body.

13. The coupling device of claim 9, wherein the first opening of the housing comprises a first cross-sectional width, and an outer surface of the wall of the inner body comprises a second cross-sectional width, and wherein the first cross-sectional width is equal to or greater than the second cross-sectional width, such that, the first end portion of the inner body is longitudinally movable between the first and second positions through the first opening.

14. The coupling device of claim 9, wherein the housing comprise a ledge that extends radially inward from the inner surface into the cavity.

15. The coupling device of claim 14, wherein, in the first position, a portion of the inner body is engaged against the ledge, thereby resisting movement of inner body toward the second end of the housing.

16. The coupling device of claim 9, wherein a distance from the first end of the inner body to a second end of the inner body is greater than a distance from the first end of the housing to a distal end of the cavity.

17. The coupling device of claim 9, further comprising a cap coupled to the first end of the housing, the cap forming at least a portion of the first opening of the housing.

18. The coupling device of claim 9, wherein the biasing element comprises a spring configured to direct the inner body toward the first position.

19. A method for providing a coupling device for medical tubing, the method comprising:
providing an inner body in a cavity of a housing such that the housing and the inner body are movable relative to each other, and a coupling sleeve defined by a proximal portion of the inner body extends through a first opening at a first end of the housing;
providing a biasing element in the housing such that the biasing element is positioned between a first end of the housing and the inner body;
providing a tubing through a second opening at a second end of the housing and couplable to a fluid passage extending through the inner body;
wherein, in a first position of the inner body, a first end of the inner body is longitudinally aligned with the first end of the housing such that biasing of at least a portion of coupling sleeve away from a longitudinal axis of the fluid passage of the inner body is resisted by the housing, and in a second position of the inner body, the second end of the housing is moved away from the second end of the inner body such that the first end of the inner body is longitudinally spaced apart from the first end of the housing and the at least a portion of the coupling sleeve can be biased away from the longitudinal axis.

20. The method of claim 19 wherein, when the inner body is in the second position and a mating connector, coupled to the coupling sleeve, is moved in a direction away from the first end of the inner body, engagement of the mating connector with the coupling sleeve directs at least a portion of the coupling sleeve away from the longitudinal axis.
